SEQUENCE LISTING (20 sequences)

SEQ ID NO: 1 - DNA, 4180 bp, unidentified
  misc_feature 1..4180: note = Description of Unknown: mRNA transcript encoding furin sequence
  source 1..4180: mol_type = genomic DNA
gcggggaagcagcagcggccaggatgaatcccaggtgctctggagctggatggtgaaggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccag

SEQ ID NO: 2 - RNA, 203 nt, synthetic construct
  misc_feature 1..203: note = Description of Sequence: Insert encoding a bi-functional short hairpin RNA
  source 1..203: mol_type = other RNA
ggatcctgctgttgacagtgagcgcggagaaaggagtgaaaccttagtgaagccacagatgtaaggtttcactcctttctccttgcctactgcctcggagtcctgctgttgacagtgagcgcggagaaagatatgaaaccttagtgaagccacagatgtaaggtttcactcctttctccttgcctactgcctcggaagctttg

SEQ ID NO: 3 - RNA, 245 nt, synthetic construct
  misc_feature 1..245: note = Description of Sequence: Insert encoding a bi-functional short hairpin RNA
  source 1..245: mol_type = other RNA
gatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatc

SEQ ID NO: 4 - DNA, 5140 bp, synthetic construct
  misc_feature 1..5140: note = Description of Sequence: plasmid
  source 1..5140: mol_type = other DNA
ggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctaaaaaaaaaaaaaaaaaaaaaaacggaattcacgtgggcccggtaccgtatactctagaagatctggcagcggagagggcagaggaagtcttctaacatgcggtgacgtggaggagaatcccggccctaggatgtctagagcggccgcggatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctatt

SEQ ID NO: 5 - DNA, 788 bp, Homo sapiens
  source 1..788: mol_type = genomic DNA
agtacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttg

SEQ ID NO: 6 - RNA, 15 nt, synthetic construct
  misc_feature 1..15: note = Description of Sequence: component of miR-30a loop
  source 1..15: mol_type = other RNA
gtgaagccacagatg

SEQ ID NO: 7 - RNA, 21 nt, synthetic construct
  misc_feature 1..21: note = Description of Sequence: stem-loop structure
  source 1..21: mol_type = other RNA
taaggtttcactcctttctcc

SEQ ID NO: 8 - RNA, 21 nt, synthetic construct
  misc_feature 1..21: note = Description of Sequence: stem-loop structure
  source 1..21: mol_type = other RNA
ggagaaaggagtgaaacctta

SEQ ID NO: 9 - RNA, 21 nt, synthetic construct
  misc_feature 1..21: note = Description of Sequence: stem-loop structure
  source 1..21: mol_type = other RNA
ggagaaagatatgaaacctta

SEQ ID NO: 10 - DNA, 22 bp, synthetic construct
  misc_feature 1..22: note = Description of Sequence: Synthetic primer
  source 1..22: mol_type = other DNA
caccattggcaatgagcggttc

SEQ ID NO: 11 - DNA, 22 bp, synthetic construct
  misc_feature 1..22: note = Description of Sequence: Synthetic primer
  source 1..22: mol_type = other DNA
aggtctttgcggatgtccacgt

SEQ ID NO: 12 - DNA, 30 bp, synthetic construct
  misc_feature 1..30: note = Description of Sequence: Synthetic primer
  source 1..30: mol_type = other DNA
cctactaaattaaatgatctctgctttact

SEQ ID NO: 13 - DNA, 21 bp, synthetic construct
  misc_feature 1..21: note = Description of Sequence: Synthetic primer
  source 1..21: mol_type = other DNA
caagctataacgcagcctgta

SEQ ID NO: 14 - DNA, 22 bp, synthetic construct
  misc_feature 1..22: note = Description of Sequence: Synthetic primer
  source 1..22: mol_type = other DNA
gccacatgactactccgcagat

SEQ ID NO: 15 - DNA, 22 bp, synthetic construct
  misc_feature 1..22: note = Description of Sequence: Synthetic primer
  source 1..22: mol_type = other DNA
tacgagggtgaacttggtcagc

SEQ ID NO: 16 - protein, 7 aa, unidentified
  REGION 1..7: note = Description of Unknown: furin cleavage site sequence
PRRARSV

SEQ ID NO: 17 - protein, 4 aa, unidentified
  REGION 1..4: note = Description of Unknown: furin-like cleavage site sequence
KRSF

SEQ ID NO: 18 - RNA, 223 nt, synthetic construct
  misc_feature 1..223: note = Description of Sequence: Artificial polynucleotide
  source 1..223: mol_type = other RNA
gatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaag

SEQ ID NO: 19 - protein, 4 aa, unidentified
  REGION 1..4: note = Description of Unknown: S1/S2 boundary sequence
PRRA

SEQ ID NO: 20 - protein, 4 aa, unidentified
  REGION 1..4: note = Description of Unknown: furin cleavage site sequence
RRAR

(19) United States
(12) Patent Application Publication
(10) Pub. No.: US 2023/0144927 A1
(43) Pub. Date: May 11, 2023

(54) METHODS FOR THE TREATMENT OF VIRAL RESPIRATORY INFECTIONS

(71) Applicant: GRADALIS, INC., Carrollton, TX (US)

(72) Inventors: DAVID SHANAHAN, Carrollton, TX (US); JOHN NEMUNAITIS, Carrollton, TX (US); ERNEST BOGNAR, Carrollton, TX (US)

(73) Assignee: GRADALIS, INC., Carrollton, TX (US)

(21) Appl. No.: 17/977,979

(22) Filed: Oct. 31, 2022

Related U.S. Application Data
(63) Continuation of application No. PCT/US21/33848, filed on May 24, 2021.
(60) Provisional application No. 63/030,214, filed on May 26, 2020.

(51) Int. Cl. C07K 14/535; C12N 15/52; C12N 15/63; A61P 31/14

(52) U.S. Cl. CPC C07K 14/535; C12N 15/52; C12N 15/63; A61P 31/14; C12N 2310/122

(57) ABSTRACT

Compositions and methods for treatment of a respiratory infection by a virus are disclosed herein. In some embodiments, the composition comprises pulmonary administration of a plasmid encoding a GM-CSF sequence and a bifunctional shRNA capable of inhibiting furin expression. In some embodiments, the virus is SARS-CoV-2.

### CROSS-REFERENCES TO RELATED APPLICATIONS

This application is a continuation of PCT/US2021/033848, filed May 24, 2021, which application claims priority to U.S. Provisional Patent Application No. 63/030,214, filed May 26, 2020, the contents both of which are hereby incorporated by reference in their entireties for all purposes.

### BACKGROUND

Coronaviruses are a family of viruses that can cause illnesses such as the common cold, severe acute respiratory syndrome (SARS) and Middle East respiratory syndrome (MERS). SARS was first reported in Asia in 2003. The illness spread to more than two dozen countries in North America, South America, Europe, and Asia before the SARS global outbreak of 2003 was contained. Symptoms of SARS are similar to cold and influenza-like symptoms, for example, an individual infected with SARS typically exhibits symptoms such as high fever, body aches, headache, dry cough, chills, fatigue or malaise, diarrhea, dyspnea (shortness of breath), and hypoxaemia (low blood oxygen concentration). To treat individuals infected with SARS, remedies to treat the upper and/or lower respiratory tract areas have been suggested. Suitable treatment methods include vaccinations against SARS, and the administration of antibiotics or antiviral drugs such as ribavirin, tetracycline, erythromycin, and corticosteroids (e.g., methylprednisolone).

Middle East Respiratory Syndrome (MERS) is a viral respiratory illness also caused by a coronavirus. It was first reported in Saudi Arabia in 2012 and spread to several other countries, including the United States. Most people infected with MERS-CoV developed severe respiratory illness, including fever, cough, and shortness of breath. Many of them have died. MERS-CoV has spread from ill people to others through close contact, such as caring for or living with an infected person.

In 2019, a new coronavirus was identified as the cause of a disease outbreak that originated in China. The virus is known as the severe acute respiratory syndrome coronavirus 2 (SARS-CoV-2). The disease it causes is called coronavirus disease 2019 (COVID-19). COVID-19 has caused a world pandemic causing seve6 disease and death in millions of infected individuals.

Given the ability of coronaviruses to cause severe respiratory illnesses, it is important to find ways to treat respiratory illnesses caused by these viruses. The present disclosure satisfies this need and offers other advantages as well.

### BRIEF SUMMARY

Disclosed herein, in certain embodiments, are methods of treating a respiratory condition caused by a virus in an individual in need thereof, the method comprising administering to the individual an expression vector comprising: a. a first insert comprising a nucleic acid sequence encoding a Granulocyte Macrophage Colony Stimulating Factor (GM-CSF) sequence; and b. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs, wherein the virus uses a furin produced by the individual to enable infection of a cell of the individual by the virus, and wherein the administering of the expression vector treats the respiratory condition. In some embodiments, administering the expression vector reduces or eliminates propagation of the virus causing the respiratory condition. In some embodiments, the second insert comprises a nucleic acid sequence according to SEQ ID NO: 2 or SEQ ID NO: 3. In some embodiments, the individual is a human. In some embodiments, the expression vector is a plasmid.

In some embodiments, the expression vector is lyophilized with at least one stabilizing excipient prior to the administering, thereby producing lyophilized particles. In some embodiments, at least one stabilizing excipient is trehalose. In some embodiments, the lyophilized particles are less than 5 μm in diameter. In some embodiments, the lyophilized particles are from about 1 μm to 3 μm in diameter. In some embodiments, from about 1 mg to about 4 mg of the expression vector is administered to the individual. In some embodiments, the administering comprises pulmonary delivery. In some embodiments, the administering comprises pulmonary delivery of the expression vector to the individual via a device selected from an inhaler or a nebulizer.

In some embodiments, the virus comprises a glycoprotein requiring cleavage by the furin to allow entry of the virus into the cell of the individual. In some embodiments, the cell is an alveolar cell. In some embodiments, the virus causing the respiratory condition is a coronavirus. In some embodiments, the coronavirus is Severe Acute Respiratory Syndrome Coronavirus-2 (SARS-CoV-2), SARS-CoV or MERS-CoV. In some embodiments, the respiratory condition is Coronavirus disease 2019 (COVID-19).

In some embodiments, the GM-CSF is a human GM-CSF sequence. In certain embodiments, the GM-CSF is a human GM-CSF sequence having SEQ ID NO: 5. In some embodiments, the expression vector further comprises a promoter. In some embodiments, the promoter is a cytomegalovirus (CMV) mammalian promoter. In some embodiments, the expression vector further comprises a CMV enhancer sequence and a CMV intron sequence. In some embodiments, the first insert and the second insert are operably linked to the promoter. In some embodiments, the expression vector further comprises a nucleic acid sequence encoding a picornaviral 2A ribosomal skip peptide between the first and the second nucleic acid inserts.

Disclosed herein, in certain embodiments, are inhalable dosage forms, comprising: a. an expression vector comprising i. a first insert comprising a nucleic acid sequence encoding a Granulocyte Macrophage Colony Stimulating Factor (GM-CSF) sequence; and ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and b. at least one stabilizing excipient. In some embodiments, the second insert comprises a nucleic acid sequence according to SEQ ID NO: 2 or SEQ ID NO: 3. In some embodiments, the at least one stabilizing excipient is trehalose. In some embodiments, the expression vector is a plasmid.

In some embodiments, the inhalable dosage form comprises particles comprising the expression vector and the at least one stabilizing excipient. In some embodiments, the particles are less than 5 μm in diameter. In some embodiments, the particles are from about 1 μm to 3 μm in diameter. In some embodiments, the particles are lyophilized particles. In some embodiments, the GM-CSF is a human GM-CSF sequence. In some embodiments, the expression vector further comprises a promoter. In some embodiments, the promoter is a cytomegalovirus (CMV) mammalian promoter. In some embodiments, the expression vector further comprises a CMV enhancer sequence and a CMV intron sequence. In some embodiments, the first insert and the second insert are operably linked to the promoter. In some embodiments, the expression vector further comprises a nucleic acid sequence encoding a picornaviral 2A ribosomal skip peptide between the first and the second nucleic acid inserts.

In some embodiments, the lyophilized composition is formulated for pulmonary delivery. In some embodiments, the lyophilized composition is formulated for pulmonary delivery via a device. In some embodiments, the device is an inhaler or a nebulizer.

Disclosed herein, in certain embodiments, are methods of generating a lyophilized composition, the method comprising: a. generating a liquid composition comprising: i. an expression vector comprising (a) a first insert comprising a nucleic acid sequence encoding a Granulocyte Macrophage Colony Stimulating Factor (GM-CSF) sequence; and (b) a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and ii. at least one stabilizing excipient; and b. lyophilizing the liquid composition from step a. using thin film freezing to generate lyophilized particles. In some embodiments, the second insert comprises a nucleic acid sequence according to SEQ ID NO: 2 or SEQ ID NO: 3. In some embodiments, the at least one stabilizing excipient is trehalose.

In some embodiments, the liquid composition comprises from about 3% to 7% w/v of the at least one stabilizing excipient. In some embodiments, the liquid composition comprises about 5% w/v of the at least one stabilizing excipient. In some embodiments, the expression vector is a plasmid. In some embodiments, the lyophilized particles are less than 5 μm in diameter. In some embodiments, the lyophilized particles are from about 1 μm to 3 μm in diameter.

In some embodiments, the GM-CSF is a human GM-CSF sequence. In some embodiments, the expression vector further comprises a promoter. In some embodiments, the promoter is a cytomegalovirus (CMV) mammalian promoter. In some embodiments, the expression vector further comprises a CMV enhancer sequence and a CMV intron sequence. In some embodiments, the first insert and the second insert are operably linked to the promoter. In some embodiments, the expression vector further comprises a nucleic acid sequence encoding a picornaviral 2A ribosomal skip peptide between the first and the second nucleic acid inserts.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a schematic showing the bi-shRNA^{furin} (SEQ ID NO:2) comprising two stem-loop structures with miR-30a backbone; the first stem-loop structure has complete complementary guiding strand and passenger strand, while the second stem-loop structure has three basepair (bp) mismatches at positions 9 to 11 of the passenger strand.

FIG. 1B shows a schematic showing the bi-shRNA^{furin} comprising the two stem-loop structures with a miR-30a backbone of SEQ ID NO:3; wherein the first stem-loop structure has complete complementary guiding strand and passenger strand, while the second stem-loop structure has three basepair (bp) mismatches at positions 9 to 11 of the passenger strand.

FIG. 2 depicts a schematic showing a 5140 base pair (bp) (SEQ ID NO: 4) plasmid containing a GM-CSF gene for expression and bifunctional furin sh-RNA, along with a kanamycin cassette and CMV promoter.

FIG. 3 shows a lyophilization procedure wherein an expression vector is in solution, and can be rapidly frozen and lyophilized. After lyophilization, the lyophilized powder becomes a dry powder. A dry powder can be aerosolized in a dry powder inhaler.

FIGS. 4A-4C show knockdown of furin reduces SARS-COV-2 viral RNA using contemporary 72B/CA/CALG strain.

FIGS. 5A-5D show the antiviral effects of lyophilized plasmid.

### DETAILED DESCRIPTION

Unless defined otherwise, all technical and scientific terms used herein have the same meaning as is commonly understood by one of skill in the art to which the claimed subject matter belongs.

### Definitions

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the autologous tumor cell vaccine required to provide a clinically significant decrease in disease symptoms without undue adverse side effects. In another example, an “effective amount” for therapeutic uses is the amount of the autologous tumor cell vaccine as disclosed herein required to prevent a relapse of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the autologous tumor cell vaccine, age, weight, general condition of the subject, the condition being treated, the severity of the condition being treated, and the judgment of the prescribing physician.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing relapse of the disease or condition, or inhibiting the symptoms of the disease or condition either prophylactically and/or therapeutically.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein the term “dry powder” refers to a fine particulate composition, with the particles capable of being borne by a stream of air or gas, the dry powder not being suspended or dissolved in a propellant, carrier or other liquid. “Dry powder” does not necessarily imply the complete absence of water molecules from the formulation. In certain instances, the dry powder is a lyophilized particle or plurality of particles.

As used herein the term “aerosol” or “aerosolized” is meant to refer to dispersions in air of solid or liquid particles. In general, such particles have low settling velocities and relative airborne stability. In certain aspects, the particle size distribution is between 0.01 μm and 15 μm.

By “agent” is meant any small molecule chemical compound, antibody, nucleic acid molecule, or polypeptide, or fragments thereof. A dry powder may be aerosolized using conventional dry powder inhalers.

### Inhalable Dosage Forms

The present disclosure provides inhalable dosage forms, comprising: a. an expression vector comprising i. a first insert comprising a nucleic acid sequence encoding a Granulocyte Macrophage Colony Stimulating Factor (GM-CSF) sequence; and ii. a second insert comprising a nucleic acid sequence encoding at least one bi-functional short hairpin RNA (bi-shRNA) capable of hybridizing to a region of a mRNA transcript encoding furin, to inhibit furin expression via RNA interference wherein the bi-shRNA incorporates cleavage dependent siRNA and cleavage independent miRNA motifs; and b. at least one stabilizing excipient.

Compositions and methods are disclosed for delivering dry powder formulations containing polynucleotides into a subject's respiratory tract including the lungs. The methods find use in the delivery of nucleic acid (e.g. DNA and RNA) expression vectors into airway epithelial cells, alveoli pulmonary macrophages and other cells in the respiratory tract (including the oropharynx nose nasopharynx). RNA polynucleotides may include shRNA, siRNA, miRNA and combinations thereof. In some embodiments, administration of particles comprising an expression vector encoding a bi-functional short hairpin RNA capable of hybridizing to a region of an mRNA transcript encoding furin, inhibits furin expression via RNA interference, and prevents or decreases propagation of the virus.

In one aspect, the inhalable dosage particles are made using methods to produce stable micron and submicron particles comprising an expression vector. In certain aspects, the methods use a thin film freezing (TFF) technique (see, for example, U.S. Pat. No. 10,092,512). In TFF, liquid droplets typically fall from a given height and impact, spread, and freeze on a cooled solid substrate. In operation, a droplet falls from a given height, and impacts a spinning surface having a temperature of 223 K. As the droplet spreads out, a freezing front is formed in advance of the unfrozen liquid. TFF can be used to form high specific surface area powder of poorly water soluble drugs. TFF can be used for forming high surface area expression vector particles. TFF dry powder formulations can be delivered directly to the lungs via an inhaler.

Dry powder formulations typically comprise the expression vector in a dry, usually lyophilized form with a particle size within a range for deposition within the alveolar region of the lung, typically having a diameter of from about 0.5 μm to about 15 μm or 0.5 μm to about 5 μm. Respirable powders containing an expression vector within the size range can be produced by a variety of conventional techniques, such as lyophilization, thin film freezing, jet-milling, spray-drying, solvent precipitation, and the like. Dry powders can then be administered to the patient or subject in conventional dry powder inhalers (DPI's) that use the patient's inspiratory breath through the device to disperse the powder or in air-assisted devices that use an external power source to disperse the powder into an aerosol cloud.

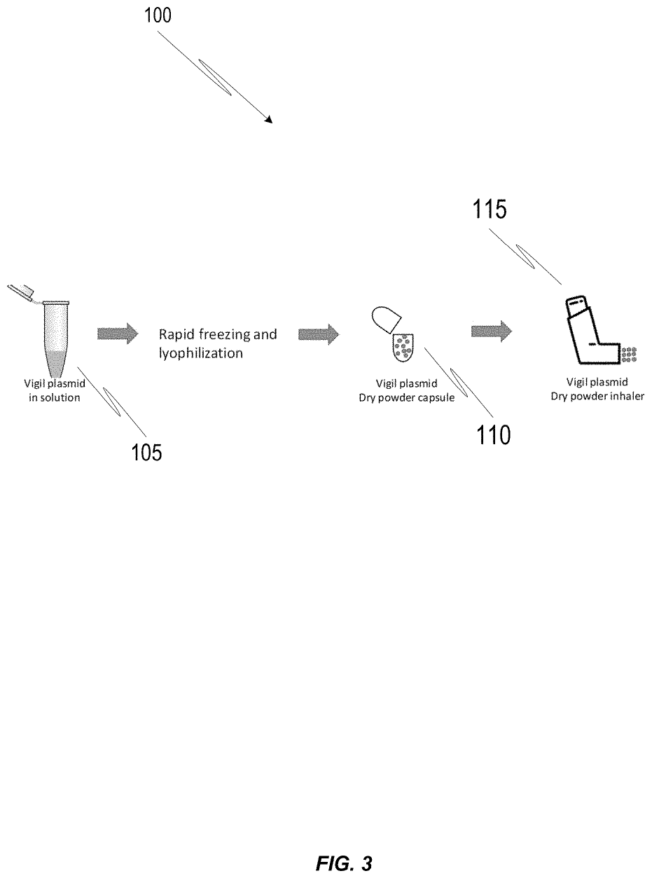

FIG. 3 shows a lyophilization method 100, wherein the expression vector is in solution 105, which can be rapidly frozen and lyophilized. After lyophilization, the lyophilized powder becomes a dry powder 110. A dry powder inhaler 115 can aerosolize the dry powder. Those skilled in the art will appreciate that lyophilization is a freeze-drying process in which water is sublimed from the composition after it is frozen. The particular advantage associated with the lyophilization process is that biologicals in an aqueous solution can be dried without elevated temperatures (thereby eliminating the adverse thermal effects), and then stored in a dry state where there are few stability problems. The lyophilized cake containing the expression vectors can be micronized using techniques known in the art to provide about 1 μm to about 10 μm, or 0.5 μm to about 5 μm sized particles.

Dry powder devices typically require a powder mass in the range from about 1 mg to 10 mg to produce a single aerosolized dose (“puff”). Since the required dose of the expression vector will generally be lower than this amount, the powder will typically be combined with a pharmaceutically acceptable dry bulking powder or one or more stabilizing excipient(s). Dry bulking powders or stabilizing excipients include sucrose, lactose, trehalose, human serum albumin (HSA), and glycine. Other suitable dry bulking powders include cellobiose, dextrans, maltotriose, pectin, sodium citrate, sodium ascorbate, mannitol, and the like. Other stabilizing excipients include glucose, arabinose, maltose, saccharose, dextrose and/or a polyalcohol such as mannitol, maltitol, lactitol and sorbitol. In one embodiment, the sugar is trehalose. In some instances, suitable buffers and salts may be used to stabilize the expression vector in solution prior to particle formation. Suitable buffers include phosphate, citrate, acetate, and tris-HCl, typically at concentrations from about 5 mM to 50 mM. Suitable salts include sodium chloride, sodium carbonate, calcium chloride, and the like.

In one aspect, the expression vector is lyophilized with at least one stabilizing excipient prior to the administering, thereby producing lyophilized particles of about 0.5 μm to about 15 μm such as about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, and/or 15 μm. In some aspects, at least one stabilizing excipient is trehalose. In some aspects, the lyophilized particles are less than 5 μm in diameter. In some aspects, the lyophilized particles are from about 1 μm to about 3 μm in diameter. In some aspects, from about 1 mg to about 4 mg of the expression vector is administered to the individual. In some aspects, the administering comprises pulmonary delivery. In some aspects, the administering comprises pulmonary delivery of the expression vector to the individual or subject via a device selected from an inhaler or a nebulizer.

In one aspect, the frequency of dosing of the expression vector can be 1-10 times daily, such a 1, 2, 3, 4, 5 6, 7, 8, 9, or 10 times per day or even more. The regimen can be over days such as 1-7 days or weeks 1-4 weeks or months such as 1-12 months.

Dry powder aerosol compositions of the present disclosure can be used to transport polynucleotides via the lung into tumors, lymph, blood and macrophages or other cells of the body. In the methods of the present disclosure, delivery is generally achieved by controlling the size of the aerosolized particle containing an expression vector. In some aspects, methods are provided for delivering a dry powder aerosolized polynucleotide to the deep lung, i.e., the alveoli. In these aspects, a majority of the aerosolized, expression vector-containing particles have a size in the range of about 0.01 μm to about 10 μm such as 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.1 μm, 4.2 μm, 4.3 μm, 4.4 μm, 4.5 μm, 4.6 μm, 4.7 μm, 4.8 μm, 4.9 μm, 5 μm, 5.1 μm, 5.2 μm, 5.3 μm, 5.4 μm, 5.5 μm, 5.6 μm, 5.7 μm, 5.8 μm, 5.9 μm, 6 μm, 6.1 μm, 6.2 μm, 6.3 μm, 6.4 μm, 6.5 μm, 6.6 μm, 6.7 μm, 6.8 μm, 6.9 μm, 7 μm, 7.1 μm, 7.2 μm, 7.3 μm, 7.4 μm, 7.5 μm, 7.6 μm, 7.7 μm, 7.8 μm, 7.9 μm, 8 μm, 8.1 μm, 8.2 μm, 8.3 μm, 8.4 μm, 8.5 μm, 8.6 μm, 8.7 μm, 8.8 μm, 8.9 μm, 9 μm, 9.1 μm, 9.2 μm, 9.3 μm, 9.4 μm, 9.5 μm, 9.6 μm, 9.7 μm, 9.8 μm, 9.9 μm, and/or about 10 μm.

In some aspects, methods are provided for delivering an aerosolized dry powder expression vector polynucleotide to the central airways, i.e., the bronchi and bronchioles. In these aspects, a majority of the dry powder aerosol, polynucleotide-containing particles have a size in the range of about 4 μm to about 6 μm (4 μm-6 μm) or about 4 μm-about 5 μm (4 μm-5 μm). In still other aspects, methods are provided for delivering aerosolized particles to the upper respiratory tract, including the oropharyngeal region and the trachea. In certain aspects, the aerosol can be delivered to the alveoli if delivery to the circulatory system is desired. In this aspect, the particle size can be about 1 to about 5 microns, and can be generally a spherical shape.

Typically, the aerosol is created by forcing the drug formulation through a nozzle comprised of a porous membrane having pores in the range of about 0.25 to 6.0 microns in size. When the pores have this size the droplets that are formed will have a diameter about twice the diameter of the pore size. In order to ensure that the low resistance filter has the same or less flow resistance as the nozzle, the pore size and pore density of the filter should be adjusted as necessary with adjustments in pore size and pore density of the nozzle's porous membrane. Particle size can also be adjusted by adding heat to evaporate carrier. From the period of time from the formation of the aerosolized particles until the particles actually contact the lung surface, the size of the particles is subject to change due to increases or decrease in the amount of water in the formulation due to the relative humidity within the surrounding atmosphere.

In certain aspects, the term “carrier” means the material which forms the particle that contains the polynucleotide or plasmid being administered, along with other excipients, including bulk media, required for the safe and efficacious action of the polynucleotide. These carriers may be dissolved, dispersed or suspended in bulk media such as water, ethanol, saline solutions and mixtures thereof. Other bulk media can also be used provided that they can be formulated to create a suitable aerosol and do not adversely affect the active component or human lung tissue. Useful bulk media do not adversely interact with the polynucleotide and have properties which allow for the formation of aerosolized particles having a diameter in the range of 1.0 to 10 microns (0.1 to 10 microns) when a formulation comprising the bulk media.

For aqueous solutions, the polynucleotides may be dissolved in water or a buffer and formed into small particles to create an aerosol which is delivered to the subject. Alternatively, the polynucleotide may be in a solution or a suspension wherein a low-boiling point propellant is used as a carrier fluid. Suitable aerosol propellants include, but are not limited to, chlorofluorocarbons (CFC) and hydrofluorocarbons (HFC), a variety of which are known in the art. The polynucleotide may be in the form of a dry powder which is intermixed with an airflow in order to provide for delivery of polynucleotide to the subject. Respirable dry powders within the desired size range can be produced by a variety of conventional techniques, including jet-milling, spray-drying, solvent precipitation, and the like.

Dry powders are generally combined with a pharmaceutically acceptable dry bulking powder, with the polynucleotide or plasmid present usually at from about 1% to about 10% such as about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, and/or 10% or even more by weight. Examples of dry bulking powders include sucrose, lactose, trehalose, human serum albumin (HSA), and glycine. Other suitable dry bulking powders include cellobiose, dextrans, maltotriose, pectin, sodium citrate, sodium ascorbate, mannitol, and the like. Regardless of the formulation, it is preferable to create particles having a size in the desired range, which is related to airway diameter of the targeted region.

In certain instances, the dry powders for inhalation are formulated as pharmaceutically active substances with carrier particles of inert material such as lactose. The carrier particles are designed such that they have a larger diameter than the active substance particles making them easier to handle and store. The smaller active agent particles are bound to the surface of carrier particles during storage, but are torn from the carrier particles upon actuation of the device.

In some aspects, the polynucleotide and expression vector to be delivered can be formulated as a liposome or lipoplex formulation. Such complexes comprise a mixture of lipids which bind to genetic material (DNA or RNA) by means of cationic charge (electrostatic interaction). Cationic liposomes which may be used in the present invention include 3β-[N—(N′, N′-dimethyl-aminoethane)-carbarnoyl]-cholesterol (DC-Chol),1,2-bis(oleoyloxy-3-trimethylammonio-propane (DOTAP), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine. Other suitable phospholipids which may be used include phosphatidylcholine, phosphatidylserine, phosphatidylethanolamine, sphingomyelin, phosphatidylinositol, and the like. Cholesterol may also be included.

### Methods of Treating Viral Respiratory Infections

Disclosed herein, in certain embodiments, are methods of treating a respiratory condition caused by a virus in an individual in need thereof, the method comprising: administering to the individual an expression vector comprising (a) a first insert comprising a nucleic acid sequence encoding a Granulocyte Macrophage Colony Stimulating Factor (GM-CSF) sequence; and (b) a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs, and wherein the administering of the expression vector treats the respiratory condition. In some embodiments, the virus uses a furin produced by the individual to enable infection of a cell of the individual by the virus. In some embodiments, the second nucleic acid comprises a sequence according to SEQ ID NO: 2 or SEQ ID NO: 3. In some embodiments, the expression vector is a bishRNA^{furin}/GMCSF expression vector. In some embodiments, the expression vector is a plasmid.

In some embodiments, the virus comprises a glycoprotein. In some embodiments, the glycoprotein is a spike glycoprotein. In some embodiments, the glycoprotein is used for viral entry, protein assembly, and viral egress into and out of human cells. In some embodiments, the glycoprotein requires cleavage by a host furin protein to enable fusion sequence activation necessary for host cell entry. In some embodiments, furin inhibition prevents propagation of the virus. In some embodiments, administration of a furin inhibition prevents propagation of the virus. In some embodiments, administration of an expression vector encoding a bi-functional short hairpin RNA capable of hybridizing to a region of an mRNA transcript encoding furin, thereby inhibiting furin expression via RNA interference, prevents or decreases propagation of the virus comprising the glycoprotein.

In some embodiments, the virus is Severe Acute Respiratory Syndrome Coronavirus-2 (SARS-CoV-2), SARS-CoV or MERS-CoV. In some embodiments, the respiratory condition is Coronavirus disease 2019 (COVID-19).

Expression Vector

In some embodiments, the at least one shRNA is at least one bifunctional shRNA (bi-shRNA). In some embodiments, the bi-shRNA comprises a first stem-loop structure that comprises an siRNA component and a second stem-loop structure that comprises a miRNA component. In some embodiments, the bi-functional shRNA has two mechanistic pathways of action, that of the siRNA and that of the miRNA. Thus, in some embodiments, the bi-functional shRNA described herein is different from a traditional shRNA, i.e., a DNA transcription derived RNA acting by the siRNA mechanism of action or from a “doublet shRNA” that refers to two shRNAs, each acting against the expression of two different genes but in the traditional siRNA mode. In some embodiments, the bi-shRNA incorporates siRNA (cleavage dependent) and miRNA (cleavage-independent) motifs.

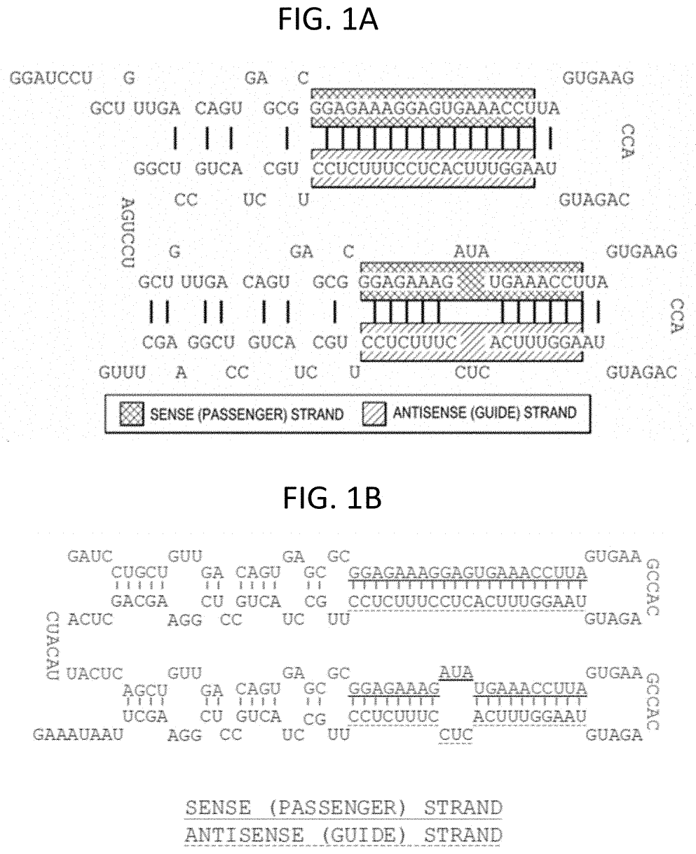

In some embodiments, the at least one bi-shRNA is capable of hybridizing to one of more regions of an mRNA transcript encoding furin. In some embodiments, the mRNA transcript encoding furin is a nucleic acid sequence of SEQ ID NO:1. In some embodiments, the one or more regions of the mRNA transcript encoding furin is selected from base sequences 300-318, 731-740, 1967-1991, 2425-2444, 2827-2851 and 2834-2852 of SEQ ID NO: 1. In some embodiments, the expression vector targets the coding region of the furin mRNA transcript, the 3′ UTR region sequence of the furin mRNA transcript, or both the coding sequence and the 3′ UTR sequence of the furin mRNA transcript simultaneously. In some embodiments, the bi-shRNA comprises SEQ ID NO: 2 or SEQ ID NO: 3. In some embodiments, a bi-shRNA capable of hybridizing to one or more regions of an mRNA transcript encoding furin is referred to herein as bi-shRNA^{furin}. In some embodiments, the bi-shRNA^{furin} comprises or consists of two stem-loop structures with miR-30a backbone. In some embodiments, a first stem-loop structure of the two stem-loop structures comprises complementary guiding strand and passenger strand (FIG. 1A or FIG. 1B). In some embodiments, the second stem-loop structure of the two stem-loop structures comprises three mismatches in the passenger strand. In some embodiments, the three mismatches are at positions 9 to 11 in the passenger strand.

In certain embodiments, the inhalable composition comprises a. an expression vector comprising i. a first insert comprising a nucleic acid sequence encoding a Granulocyte Macrophage Colony Stimulating Factor (GM-CSF) sequence; and ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and b. at least one stabilizing excipient.

In some embodiments, the GM-CSF in the expression vector is a human GM-CSF sequence. In some embodiments, the expression vector further comprises a promoter, e.g., the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the expression vector plasmid can have a sequence that is at least 90% (e.g., 90%, 91%, 92%, 93%, 94%, 95%, 96%, 97%, 98%, 99%, or 100%) identical to the sequence of SEQ ID NO:4. The vector plasmid can comprise a first nucleic acid insert operably linked to a promoter, wherein the first insert encodes the GM-CSF cDNA, a second nucleic acid insert operably linked to the promoter, wherein the second insert encodes one or more short hairpin RNAs (shRNA) capable of hybridizing to a region of a mRNA transcript encoding furin, thereby inhibiting furin expression via RNA interference.

In SEQ ID NO: 4, the bolded underlined portion is the GM-CSF sequence and the braided underlined in the furin shRNA portion of the sequence.

An expression vector comprising a first nucleic acid encoding GM-CSF and a second nucleic acid encoding at least one bifunctional short hairpin RNA (bi-shRNA) capable of hybridizing to a region of an mRNA transcript encoding furin is referred to as a bishRNA^{furin}/GMC SF expression vector.

In certain aspects, the second insert comprises a nucleic acid sequence according to SEQ ID NO: 3.

In certain aspects, the at least one stabilizing excipient is trehalose. In some aspects, the expression vector is a plasmid.

In certain aspects, the GM-CSF is a human GM-CSF sequence.

In certain aspects, the expression vector further comprises a promoter.

In certain aspects, the promoter is a cytomegalovirus (CMV) mammalian promoter.

In certain aspects, the expression vector further comprises a CMV enhancer sequence and a CMV intron sequence.

In certain aspects, the first insert and the second insert are operably linked to the promoter.

In certain aspects, the expression vector further comprises a nucleic acid sequence encoding a picornaviral 2A ribosomal skip peptide between the first and the second nucleic acid inserts.

In some aspects, the GM-CSF is human GM-CSF. In certain instances, the first nucleic acid encoding GM-CSF is rhGM-CSF (recombinant human granulocyte-macrophage colony stimulating factor) cDNA. The accession number for Homo sapiens colony stimulating factor 2 (CSF2), mRNA is NM_000758 and is SEQ ID NO: 5. in some aspects, a nucleotide sequence encoding a picornaviral 2A ribosomal skip peptide sequence is intercalated between the first and the second nucleic acid inserts.

Disclosed herein, in certain embodiments, are methods of treating a viral infection in an individual in need thereof, the method comprising administering by inhalation to the individual, an expression vector comprising: a. a first insert comprising a nucleic acid sequence encoding a Granulocyte Macrophage Colony Stimulating Factor (GM-CSF) sequence; and b. a second insert comprising two stem-loop structures each with a miR-30a loop; the first stem-loop structure has complete complementary guiding strand and passenger strand, while the second stem-loop structure has three basepair (bp) mismatches at positions 9 to 11 of the passenger strand. Descriptions of the miR-30a loop and its sequence are known in the art, see, e.g., Rao et al., Cancer Gene Ther. 17(11):780-91, 2010; Jay et al., Cancer Gene Ther. 20(12):683-9, 2013; Rao et al., Mol Ther. 24(8):1412-22, 2016; Phadke et al., DNA Cell Biol. 30(9):715-26, 2011; Barve et al., Mol Ther. 23(6):1123-1130, 2015; Rao et al., Methods Mol Biol. 942:259-78, 2013; and Senzer et al., Mol Ther. 20(3):679-86, 2012. In some embodiments, the miR-30a loop comprises the sequence of GUGAAGCCACAGAUG (SEQ ID NO:6). In some embodiments, the guiding strand in the first stem-loop structure comprises the sequence of SEQ ID NO:7 and the passenger strand in the first stem-loop structure has the sequence of SEQ ID NO:8. In some embodiments, the guiding strand in the second stem-loop structure comprises the sequence of SEQ ID NO:7 and the passenger strand in the second stem-loop structure has the sequence of SEQ ID NO:9.

Granulocyte-macrophage colony-stimulating factor, often abbreviated to GM-CSF, is a protein secreted by macrophages, cells, mast cells, endothelial cells and fibroblasts. When integrated as a cytokine transgene, GM-CSF enhances presentation of cancer vaccine peptides, tumor cell lysates, or whole tumor cells from either autologous or established allogeneic tumor cell lines. GM-CSF induces the differentiation of hematopoietic precursors and attracts them to the site of vaccination. GM-CSF also functions as an adjuvant for dendritic cell maturation and activational processes. However, GM-CST-mediated immunosensitization can be suppressed by tumor produced and/or secreted different isoforms of transforming growth factor beta (TGF-β). The TGF-β family of multifunctional proteins possesses well known immunosuppressive activities. The three known TGF-β ligands (TGF-β1, β2, and β3) are ubiquitous in human cancers. TGF-β overexpression correlates with tumor progression and poor prognosis. Elevated TGF-β levels within the tumor microenvironment are linked to an anergic antitumor response. TGF-β inhibits GM-CSF induced maturation of dendritic cells and their expression of MHC class II and co-stimulatory molecules. This negative impact of TGF-β on GM-CST-mediated immune activation supports the rationale of depleting TGF-β secretion in GM-CSF-based cancer cell vaccines.

All mature isoforms of TGF-β require furin-mediated limited proteolytic cleavage for proper activity. Furin, a calcium-dependent serine endoprotease, is a member of the subtilisin-like proprotein convertase family. Furin is best known for the functional activation of TGF-β with corresponding immunoregulatory ramifications.

TABLE 1
Sequences

| SEQ ID | | |
|---|---|---|
| NO: | Description | Sequence |
| 1 | mRNA | GCGGGGAAGCAGCAGCGGCCAGGATGAATCCCAGGTG |
| | transcript | CTCTGGAGCTGGATGGTGAAGGTCGGCACTCTTCACCC |
| | encoding | TCCCGAGCCCTGCCCGTCTCGGCCCCATGCCCCCACCA |
| | furin | GTCAGCCCCGGGCCACAGGCAGTGAGCAGGCACCTGG |
| | | GAGCCGAGGCCCTGTGACCAGGCCAAGGAGACGGGCG |
| | | CTCCAGGGTCCCAGCCACCTGTCCCCCCCATGGAGCTG |
| | | AGGCCCTGGTTGCTATGGGTGGTAGCAGCAACAGGAA |
| | | CCTTGGTCCTGCTAGCAGCTGATGCTCAGGGCCAGAAG |
| | | GTCTTCACCAACACGTGGGCTGTGCGCATCCCTGGAGG |
| | | CCCAGCGGTGGCCAACAGTGTGGCACGGAAGCATGGG |
| | | TTCCTCAACCTGGGCCAGATCTTCGGGGACTATTACCA |
| | | CTTCTGGCATCGAGGAGTGACGAAGCGGTCCCTGTCGC |
| | | CTCACCGCCCGCGGCACAGCCGGCTGCAGAGGGAGCC |
| | | TCAAGTACAGTGGCTGGAACAGCAGGTGGCAAAGCGA |
| | | CGGACTAAACGGGACGTGTACCAGGAGCCCACAGACC |
| | | CCAAGTTTCCTCAGCAGTGGTACCTGTCTGGTGTCACTC |
| | | AGCGGGACCTGAATGTGAAGGCGGCCTGGGCGCAGGG |
| | | CTACACAGGGCACGGCATTGTGGTCTCCATTCTGGACG |
| | | ATGGCATCGAGAAGAACCACCCGGACTTGGCAGGCAA |
| | | TTATGATCCTGGGGCCAGTTTTGATGTCAATGACCAGG |
| | | ACCCTGACCCCCAGCCTCGGTACACACAGATGAATGAC |
| | | AACAGGCACGGCACACGGTGTGCGGGGGAAGTGGCTG |
| | | CGGTGGCCAACAACGGTGTCTGTGGTGTAGGTGTGGCC |
| | | TACAACGCCCGCATTGGAGGGGTGCGCATGCTGGATGG |
| | | CGAGGTGACAGATGCAGTGGAGGCACGCTCGCTGGGC |
| | | CTGAACCCCAACCACATCCACATCTACAGTGCCAGCTG |
| | | GGGCCCCGAGGATGACGGCAAGACAGTGGATGGGCCA |
| | | GCCCGCCTCGCCGAGGAGGCCTTCTTCCGTGGGGTTAG |
| | | CCAGGGCCGAGGGGGGCTGGGCTCCATCTTTGTCTGGG |
| | | CCTCGGGGAACGGGGGCCGGGAACATGACAGCTGCAA |
| | | CTGCGACGGCTACACCAACAGTATCTACACGCTGTCCA |
| | | TCAGCAGCGCCACGCAGTTTGGCAACGTGCCGTGGTAC |
| | | AGCGAGGCCTGCTCGTCCACACTGGCCACGACCTACAG |
| | | CAGTGGCAACCAGAATGAGAAGCAGATCGTGACGACT |
| | | GACTTGCGGCAGAAGTGCACGGAGTCTCACACGGGCA |
| | | CCTCAGCCTCTGCCCCCTTAGCAGCCGGCATCATTGCTC |
| | | TCACCCTGGAGGCCAATAAGAACCTCACATGGCGGGA |
| | | CATGCAACACCTGGTGGTACAGACCTCGAAGCCAGCCC |
| | | ACCTCAATGCCAACGACTGGGCCACCAATGGTGTGGGC |
| | | CGGAAAGTGAGCCACTCATATGGCTACGGGCTTTTGGA |
| | | CGCAGGCGCCATGGTGGCCCTGGCCCAGAATTGGACCA |
| | | CAGTGGCCCCCCAGCGGAAGTGCATCATCGACATCCTC |
| | | ACCGAGCCCAAAGACATCGGGAAACGGCTCGAGGTGC |
| | | GGAAGACCGTGACCGCGTGCCTGGGCGAGCCCAACCA |
| | | CATCACTCGGCTGGAGCACGCTCAGGCGCGGCTCACCC |
| | | TGTCCTATAATCGCCGTGGCGACCTGGCCATCCACCTG |
| | | GTCAGCCCCATGGGCACCCGCTCCACCCTGCTGGCAGC |
| | | CAGGCCACATGACTACTCCGCAGATGGGTTTAATGACT |
| | | GGGCCTTCATGACAACTCATTCCTGGGATGAGGATCCC |
| | | TCTGGCGAGTGGGTCCTAGAGATTGAAAACACCAGCG |
| | | AAGCCAACAACTATGGGACGCTGACCAAGTTCACCCTC |
| | | GTACTCTATGGCACCGCCCCTGAGGGGCTGCCCGTACC |
| | | TCCAGAAAGCAGTGGCTGCAAGACCCTCACGTCCAGTC |
| | | AGGCCTGTGTGGTGTGCGAGGAAGGCTTCTCCCTGCAC |
| | | CAGAAGAGCTGTGTCCAGCACTGCCCTCCAGGGTTCGC |
| | | CCCCCAAGTCCTCGATACGCACTATAGCACCGAGAATG |
| | | ACGTGGAGACCATCCGGGCCAGCGTCTGCGCCCCCTGC |
| | | CACGCCTCATGTGCCACATGCCAGGGGCCGGCCCTGAC |
| | | AGACTGCCTCAGCTGCCCCAGCCACGCCTCCTTGGACC |
| | | CTGTGGAGCAGACTTGCTCCCGGCAAAGCCAGAGCAG |
| | | CCGAGAGTCCCCGCCACAGCAGCAGCCACCTCGGCTGC |
| | | CCCCGGAGGTGGAGGCGGGGCAACGGCTGCGGGCAGG |
| | | GCTGCTGCCCTCACACCTGCCTGAGGTGGTGGCCGGCC |
| | | TCAGCTGCGCCTTCATCGTGCTGGTCTTCGTCACTGTCT |
| | | TCCTGGTCCTGCAGCTGCGCTCTGGCTTTAGTTTTCGGG |
| | | GGGTGAAGGTGTACACCATGGACCGTGGCCTCATCTCC |
| | | TACAAGGGGCTGCCCCCTGAAGCCTGGCAGGAGGAGT |
| | | GCCCGTCTGACTCAGAAGAGGACGAGGGCCGGGGCGA |
| | | GAGGACCGCCTTTATCAAAGACCAGAGCGCCCTCTGAT |
| | | GAGCCCACTGCCCACCCCCTCAAGCCAATCCCCTCCTT |
| | | GGGCACTTTTTAATTCACCAAAGTATTTTTTTATCTTGG |
| | | GACTGGGTTTGGACCCCAGCTGGGAGGCAAGAGGGGT |
| | | GGAGACTGCTTCCCATCCTACCCTCGGGCCCACCTGGC |
| | | CACCTGAGGTGGGCCCAGGACCAGCTGGGGCGTGGGG |
| | | AGGGCCGTACCCCACCCTCAGCACCCCTTCCATGTGGA |
| | | GAAAGGAGTGAAACCTTTAGGGCAGCTTGCCCCGGCCC |
| | | CGGCCCCAGCCAGAGTTCCTGCGGAGTGAAGAGGGGC |
| | | AGCCCTTGCTTGTTGGGATTCCTGACCCAGGCCGCAGC |
| | | TCTTGCCCTTCCCTGTCCCTCTAAAGCAATAATGGTCCC |
| | | ATCCAGGCAGTCGGGGGCTGGCCTAGGAGATATCTGA |
| | | GGGAGGAGGCCACCTCTCCAAGGGCTTCTGCACCCTCC |
| | | ACCCTGTCCCCCAGCTCTGGTGAGTCTTGGCGGCAGCA |
| | | GCCATCATAGGAAGGGACCAAGGCAAGGCAGGTGCCT |
| | | CCAGGTGTGCACGTGGCATGTGGCCTGTGGCCTGTGTC |
| | | CCATGACCCACCCCTGTGCTCCGTGCCTCCACCACCAC |
| | | TGGCCACCAGGCTGGCGCAGCCAAGGCCGAAGCTCTG |
| | | GCTGAACCCTGTGCTGGTGTCCTGACCACCCTCCCCTCT |
| | | CTTGCACCCGCCTCTCCCGTCAGGGCCCAAGTCCCTGTT |
| | | TTCTGAGCCCGGGCTGCCTGGGCTGTTGGCACTCACAG |
| | | ACCTGGAGCCCCTGGGTGGGTGGTGGGGAGGGGCGCT |
| | | GGCCCAGCCGGCCTCTCTGGCCTCCCACCCGATGCTGC |
| | | TTTCCCCTGTGGGGATCTCAGGGGCTGTTTGAGGATAT |
| | | ATTTTCACTTTGTGATTATTTCACTTTAGATGCTGATGA |
| | | TTTGTTTTTGTATTTTTAATGGGGGTAGCAGCTGGACTA |
| | | CCCACGTTCTCACACCCACCGTCCGCCCTGCTCCTCCCT |
| | | GGCTGCCCTGGCCCTGAGGTGTGGGGGCTGCAGCATGT |
| | | TGCTGAGGAGTGAGGAATAGTTGAGCCCCAAGTCCTGA |
| | | AGAGGCGGGCCAGCCAGGCGGGCTCAAGGAAAGGGGG |
| | | TCCCAGTGGGAGGGGCAGGCTGACATCTGTGTTTCAAG |
| | | TGGGGCTCGCCATGCCGGGGGTTCATAGGTCACTGGCT |
| | | CTCCAAGTGCCAGAGGTGGGCAGGTGGTGGCACTGAG |
| | | CCCCCCCAACACTGTGCCCTGGTGGAGAAAGCACTGAC |
| | | CTGTCATGCCCCCCTCAAACCTCCTCTTCTGACGTGCCT |
| | | TTTGCACCCCTCCCATTAGGACAATCAGTCCCCTCCCAT |
| | | CTGGGAGTCCCCTTTTCTTTTCTACCCTAGCCATTCCTG |
| | | GTACCCAGCCATCTGCCCAGGGGTGCCCCCTCCTCTCC |
| | | CATCCCCCTGCCCTCGTGGCCAGCCCGGCTGGTTTTGTA |
| | | AGATGCTGGGTTGGTGCACAGTGATTTTTTTCTTGTAAT |
| | | TTAAACAGGCCCAGCATTGCTGGTTCTATTTAATGGAC |
| | | ATGAGATAATGTTAGAGGTTTTAAAGTGATTAAACGTG |
| | | CAGACTATGCAAACCAG |
| 2 | bi-shRNA^{furin} | GGAUCCUGCUGUUGACAGUGAGCGCGGAGAAAGGAG |
| | | UGAAACCUUAGUGAAGCCACAGAUGUAAGGUUUCAC |
| | | UCCUUUCUCCUUGCCUACUGCCUCGGAGUCCUGCUGU |
| | | UGACAGUGAGCGCGGAGAAAGAUAUGAAACCUUAGU |
| | | GAAGCCACAGAUGUAAGGUUUCACUCCUUUCUCCUUG |
| | | CCUACUGCCUCGGAAGCUUUG |
| 3 | bi-shRNA^{furin} | GAUCCUGCUGUUGACAGUGAGCGCGGAGAAAGGAGU |
| | | GAAACCUUAGUGAAGCCACAGAUGUAAGGUUUCACU |
| | | CCUUUCUCCUUGCCUACUGCCUCGGAAGCAGCUCACU |
| | | ACAUUACUCAGCUGUUGACAGUGAGCGCGGAGAAAG |
| | | AUAUGAAACCUUAGUGAAGCCACAGAUGUAAGGUUU |
| | | CACUCCUUUCUCCUUGCCUACUGCCUCGGAAGCUUAA |
| | | UAAAGGAUCUUUUAUUUUCAUUGGAUC |
| 4 | Plasmid | GGCCATTGCATACGTTGTATCCATATCATAATATGTAC |
| | | ATTTATATTGGCTCATGTCCAACATTACCGCCATGTTGA |
| | | CATTGATTATTGACTAGTTATTAATAGTAATCAATTACG |
| | | GGGTCATTAGTTCATAGCCCATATATGGAGTTCCGCGT |
| | | TACATAACTTACGGTAAATGGCCCGCCTGGCTGACCGC |
| | | CCAACGACCCCCGCCCATTGACGTCAATAATGACGTAT |
| | | GTTCCCATAGTAACGCCAATAGGGACTTTCCATTGACG |
| | | TCAATGGGTGGAGTATTTACGGTAAACTGCCCACTTGG |
| | | CAGTACATCAAGTGTATCATATGCCAAGTACGCCCCCT |
| | | ATTGACGTCAATGACGGTAAATGGCCCGCCTGGCATTA |
| | | TGCCCAGTACATGACCTTATGGGACTTTCCTACTTGGC |
| | | AGTACATCTACGTATTAGTCATCGCTATTACCATGGTG |
| | | ATGCGGTTTTGGCAGTACATCAATGGGCGTGGATAGCG |
| | | GTTTGACTCACGGGGATTTCCAAGTCTCCACCCCATTG |
| | | ACGTCAATGGGAGTTTGTTTTGGCACCAAAATCAACGG |
| | | GACTTTCCAAAATGTCGTAACAACTCCGCCCCATTGAC |
| | | GCAAATGGGCGGTAGGCGTGTACGGTGGGAGGTCTAT |
| | | ATAAGCAGAGCTCGTTTAGTGAACCGTCAGATCGCCTG |
| | | GAGACGCCATCCACGCTGTTTTGACCTCCATAGAAGAC |
| | | ACCGGGACCGATCCAGCCTCCGCGGCCGGGAACGGTG |
| | | CATTGGAACGCGGATTCCCCGTGCCAAGAGTGACGTAA |
| | | GTACCGCCTATAGACTCTATAGGCACACCCCTTTGGCT |
| | | CTTATGCATGCTATACTGTTTTTGGCTTGGGGCCTATAC |
| | | ACCCCCGCTTCCTTATGCTATAGGTGATGGTATAGCTTA |
| | | GCCTATAGGTGTGGGTTATTGACCATTATTGACCACTC |
| | | CAACGGTGGAGGGCAGTGTAGTCTGAGCAGTACTCGTT |
| | | GCTGCCGCGCGCGCCACCAGACATAATAGCTGACAGA |
| | | CTAACAGACTGTTCCTTTCCATGGGTCTTTTCTGCAGTC |
| | | ACCGTCGTCGACGGTATCGATAAGCTTGATATCGAATT |
| | | CCGCTGGAGGATGTGGCTGCAGAGCCTGCTGCTCTT |
| | | GGGCACTGTGGCCTGCAGCATCTCTGCACCCGCCC |
| | | GCTCGCCCAGCCCCAGCACGCAGCCCTGGGAGCAT |
| | | GTGAATGCCATCCAGGAGGCCCGGCGTCTCCTGAA |
| | | CCTGAGTAGAGACACTGCTGCTGAGATGAATGAAAC |
| | | AGTAGAAGTCATCTCAGAAATGTTTGACCTCCAGGA |
| | | GCCGACCTGCCTACAGACCCGCCTGGAGCTGTACA |
| | | AGCAGGGCCTGCGGGGCAGCCTCACCAAGCTCAAG |
| | | GGCCCCTTGACCATGATGGCCAGCCACTACAAGCA |
| | | GCACTGCCCTCCAACCCCGGAAACTTCCTGTGCAAC |
| | | CCAGACTATCACCTTTGAAAGTTTCAAAGAGAACCT |
| | | GAAGGACTTTCTGCTTGTCATCCCCTTTGACTGCTG |
| | | GGAGCCAGTCCAGGAGTGAGACCGGCCAGATGAGG |
| | | CTGGCCAAGCCGGGGAGCTGCTCTCTCATGAAACA |
| | | AGAGCTAGAAACTCAGGATGGTCATCTTGGAGGGA |
| | | CCAAGGGGTGGGCCACAGCCATGGTGGGAGTGGCC |
| | | TGGACCTGCCCTGGGCCACACTGACCCTGATACAG |
| | | GCATGGCAGAAGAATGGGAATATTTTATACTGACAG |
| | | AAATCAGTAATATTTATATATTTATATTTTTAAAATA |
| | | TTTATTTATTTATTTATTTAAGTTCATATTCCATATTT |
| | | ATTCAAGATGTTTTACCGTAATAATTATTATTAAAAA |
| | | TATGCTTCTAAAAAAAAAAAAAAAAAAAAAAACGGA |
| | | ATTCACGTGGGCCCGGTACCGTATACTCTAGAAGATCT |
| | | GGCAGCGGAGAGGGCAGAGGAAGTCTTCTAACATGCG |
| | | GTGACGTGGAGGAGAATCCCGGCCCTAGGATGTCTAG |
| | | AGCGGCCGCGGATCCTGCTGTTGACAGTGAGCGCGGA |
| | | GAAAGGAGTGAAACCTTAGTGAAGCCACAGATGTAAG |
| | | GTTTCACTCCTTTCTCCTTGCCTACTGCCTCGGAAGCAG |
| | | CTCACTACATTACTCAGCTGTTGACAGTGAGCGCGGAG |
| | | AAAGATATGAAACCTTAGTGAAGCCACAGATGTAAGG |
| | | TTTCACTCCTTTCTCCTTGCCTACTGCCTCGGAAGCTTA |
| | | ATAAAGGATCTTTTATTTTCATTGGATCCAGATCTTTTT |
| | | CCCTCTGCCAAAAATTATGGGGACATCATGAAGCCCCT |
| | | TGAGCATCTGACTTCTGGCTAATAAAGGAAATTTATTT |
| | | TCATTGCAATAGTGTGTTGGAATTTTTTGTGTCTCTCAC |
| | | TCGGAAGGACATATGGGAGGGCAAATCATTTAAAACA |
| | | TCAGAATGAGTATTTGGTTTAGAGTTTGGCAACATATG |
| | | CCCATTCTTCCGCTTCCTCGCTCACTGACTCGCTGCGCT |
| | | CGGTCGTTCGGCTGCGGCGAGCGGTATCAGCTCACTCA |
| | | AAGGCGGTAATACGGTTATCCACAGAATCAGGGGATA |
| | | ACGCAGGAAAGAACATGTGAGCAAAAGGCCAGCAAAA |
| | | GGCCAGGAACCGTAAAAAGGCCGCGTTGCTGGCGTTTT |
| | | TCCATAGGCTCCGCCCCCCTGACGAGCATCACAAAAAT |
| | | CGACGCTCAAGTCAGAGGTGGCGAAACCCGACAGGAC |
| | | TATAAAGATACCAGGCGTTTCCCCCTGGAAGCTCCCTC |
| | | GTGCGCTCTCCTGTTCCGACCCTGCCGCTTACCGGATAC |
| | | CTGTCCGCCTTTCTCCCTTCGGGAAGCGTGGCGCTTTCT |
| | | CATAGCTCACGCTGTAGGTATCTCAGTTCGGTGTAGGT |
| | | CGTTCGCTCCAAGCTGGGCTGTGTGCACGAACCCCCCG |
| | | TTCAGCCCGACCGCTGCGCCTTATCCGGTAACTATCGT |
| | | CTTGAGTCCAACCCGGTAAGACACGACTTATCGCCACT |
| | | GGCAGCAGCCACTGGTAACAGGATTAGCAGAGCGAGG |
| | | TATGTAGGCGGTGCTACAGAGTTCTTGAAGTGGTGGCC |
| | | TAACTACGGCTACACTAGAAGAACAGTATTTGGTATCT |
| | | GCGCTCTGCTGAAGCCAGTTACCTTCGGAAAAAGAGTT |
| | | GGTAGCTCTTGATCCGGCAAACAAACCACCGCTGGTAG |
| | | CGGTGGTTTTTTTGTTTGCAAGCAGCAGATTACGCGCA |
| | | GAAAAAAAGGATCTCAAGAAGATCCTTTGATCTTTTCT |
| | | ACGGGGTCTGACGCTCAGTGGAACGAAAACTCACGTTA |
| | | AGGGATTTTGGTCATGAGATTATCAAAAAGGATCTTCA |
| | | CCTAGATCCTTTTAAATTAAAAATGAAGTTTTAAATCA |
| | | ATCTAAAGTATATATGAGTAAACTTGGTCTGACAGTTA |
| | | CCAATGCTTAATCAGTGAGGCACCTATCTCAGCGATCT |
| | | GTCTATTTCGTTCATCCATAGTTGCCTGACTCGGGGGG |
| | | GGGGGGCGCTGAGGTCTGCCTCGTGAAGAAGGTGTTGC |
| | | TGACTCATACCAGGCCTGAATCGCCCCATCATCCAGCC |
| | | AGAAAGTGAGGGAGCCACGGTTGATGAGAGCTTTGTT |
| | | GTAGGTGGACCAGTTGGTGATTTTGAACTTTTGCTTTGC |
| | | CACGGAACGGTCTGCGTTGTCGGGAAGATGCGTGATCT |
| | | GATCCTTCAACTCAGCAAAAGTTCGATTTATTCAACAA |
| | | AGCCGCCGTCCCGTCAAGTCAGCGTAATGCTCTGCCAG |
| | | TGTTACAACCAATTAACCAATTCTGATTAGAAAAACTC |
| | | ATCGAGCATCAAATGAAACTGCAATTTATTCATATCAG |
| | | GATTATCAATACCATATTTTTGAAAAAGCCGTTTCTGTA |
| | | ATGAAGGAGAAAACTCACCGAGGCAGTTCCATAGGAT |
| | | GGCAAGATCCTGGTATCGGTCTGCGATTCCGACTCGTC |
| | | CAACATCAATACAACCTATTAATTTCCCCTCGTCAAAA |
| | | ATAAGGTTATCAAGTGAGAAATCACCATGAGTGACGA |
| | | CTGAATCCGGTGAGAATGGCAAAAGCTTATGCATTTCT |
| | | TTCCAGACTTGTTCAACAGGCCAGCCATTACGCTCGTC |
| | | ATCAAAATCACTCGCATCAACCAAACCGTTATTCATTC |
| | | GTGATTGCGCCTGAGCGAGACGAAATACGCGATCGCTG |
| | | TTAAAAGGACAATTACAAACAGGAATCGAATGCAACC |
| | | GGCGCAGGAACACTGCCAGCGCATCAACAATATTTTCA |
| | | CCTGAATCAGGATATTCTTCTAATACCTGGAATGCTGTT |
| | | TTCCCGGGGATCGCAGTGGTGAGTAACCATGCATCATC |
| | | AGGAGTACGGATAAAATGCTTGATGGTCGGAAGAGGC |
| | | ATAAATTCCGTCAGCCAGTTTAGTCTGACCATCTCATCT |
| | | GTAACATCATTGGCAACGCTACCTTTGCCATGTTTCAG |
| | | AAACAACTCTGGCGCATCGGGCTTCCCATACAATCGAT |
| | | AGATTGTCGCACCTGATTGCCCGACATTATCGCGAGCC |
| | | CATTTATACCCATATAAATCAGCATCCATGTTGGAATTT |
| | | AATCGCGGCCTCGAGCAAGACGTTTCCCGTTGAATATG |
| | | GCTCATAACACCCCTTGTATTACTGTTTATGTAAGCAG |
| | | ACAGTTTTATTGTTCATGATGATATATTTTTATCTTGTG |
| | | CAATGTAACATCAGAGATTTTGAGACACAACGTGGCTT |
| | | TCCCCCCCCCCCCATTATTGAAGCATTTATCAGGGTTAT |
| | | TGTCTCATGAGCGGATACATATTTGAATGTATTTAGAA |
| | | AAATAAACAAATAGGGGTTCCGCGCACATTTCCCCGAA |
| | | AAGTGCCACCTGACGTCTAAGAAACCATTATTATCATG |
| | | ACATTAACCTATAAAAATAGGCGTATCACGAGGCCCTT |
| | | TCGTCTCGCGCGTTTCGGTGATGACGGTGAAAACCTCT |
| | | GACACATGCAGCTCCCGGAGACGGTCACAGCTTGTCTG |
| | | TAAGCGGATGCCGGGAGCAGACAAGCCCGTCAGGGCG |
| | | CGTCAGCGGGTGTTGGCGGGTGTCGGGGCTGGCTTAAC |
| | | TATGCGGCATCAGAGCAGATTGTACTGAGAGTGCACCA |
| | | TATGCGGTGTGAAATACCGCACAGATGCGTAAGGAGA |
| | | AAATACCGCATCAGATTGGCTATT |
| 5 | HUMAN | AGTACACAGAGAGAAAGGCTAAAGTTCTCTGGAGGAT |
| | GM-CSF | GTGGCTGCAGAGCCTGCTGCTCTTGGGCACTGTGGCCT |
| | SEQUENCE | GCAGCATCTCTGCACCCGCCCGCTCGCCCAGCCCCAGC |
| | | ACGCAGCCCTGGGAGCATGTGAATGCCATCCAGGAGG |
| | | CCCGGCGTCTCCTGAACCTGAGTAGAGACACTGCTGCT |
| | | GAGATGAATGAAACAGTAGAAGTCATCTCAGAAATGT |
| | | TTGACCTCCAGGAGCCGACCTGCCTACAGACCCGCCTG |
| | | GAGCTGTACAAGCAGGGCCTGCGGGGCAGCCTCACCA |
| | | AGCTCAAGGGCCCCTTGACCATGATGGCCAGCCACTAC |
| | | AAGCAGCACTGCCCTCCAACCCCGGAAACTTCCTGTGC |
| | | AACCCAGATTATCACCTTTGAAAGTTTCAAAGAGAACC |
| | | TGAAGGACTTTCTGCTTGTCATCCCCTTTGACTGCTGGG |
| | | AGCCAGTCCAGGAGTGAGACCGGCCAGATGAGGCTGG |
| | | CCAAGCCGGGGAGCTGCTCTCTCATGAAACAAGAGCTA |
| | | GAAACTCAGGATGGTCATCTTGGAGGGACCAAGGGGT |
| | | GGGCCACAGCCATGGTGGGAGTGGCCTGGACCTGCCCT |
| | | GGGCCACACTGACCCTGATACAGGCATGGCAGAAGAA |
| | | TGGGAATATTTTATACTGACAGAAATCAGTAATATTTA |
| | | TATATTTATATTTTTAAAATATTTATTTATTTATTTATTT |
| | | AAGTTCATATTCCATATTTATTCAAGATGTTTTACCGTA |
| | | ATAATTATTATTAAAAATATGCTTCTACTTG |
| 6 | MIR-30 A | GUGAAGCCACAGAUG |
| | LOOP | |
| 7 | GUIDE | UAAGGUUUCACUCCUUUCUCC |
| | STRAND IN | |
| | BOTH | |
| | FIRST AND | |
| | SECOND | |
| | STEM-LOOP | |
| | STRUCTURES | |
| | IN BI- | |
| | SHRNA^{FURIN} | |
| 8 | PASSENGER | GGAGAAAGGAGUGAAACCUUA |
| | STRAND | |
| | IN FIRST | |
| | STEM-LOOP | |
| | STRUCTURE | |
| | IN BI- | |
| | SHRNA^{FURIN} | |
| 9 | PASSENGER | GGAGAAAGAUAUGAAACCUUA |
| | STRAND | |
| | IN SECOND | |
| | STEM-LOOP | |
| | STRUCTURE | |
| | IN BI- | |
| | SHRNA^{FURIN} | |

An expression vector comprising a first nucleic acid encoding GM-CSF and a second nucleic acid encoding at least one bifunctional short hairpin RNA (bi-shRNA) capable of hybridizing to a region of an mRNA transcript encoding furin is referred to as a bishRNA^{furin}/GMCSF expression vector.

In some embodiments, the first nucleic acid and the second nucleic acid are operably linked to a promoter. In some embodiments, the promoter is a cytomegalovirus (CMV) promoter. In some embodiments, the CMV promoter is a mammalian CMV promoter. In some embodiments, the mammalian CMV promoter comprises a CMV immediate early (IE) 5′ UTR enhancer sequence and a CMV IE Intron A.

In some embodiments, the GM-CSF is human GM-CSF. In some embodiments, a nucleotide sequence encoding a picornaviral 2A ribosomal skip peptide sequence is intercalated between the first and the second nucleic acid inserts.

### EXAMPLES

### Example 1: Lyophilization Procedure to Produce Lyophilized Vigils Plasmid Particles

Anti-furin therapeutic: GM-CSF bi-shRNA^{furin} plasmid (VP) SEQ ID NO: 4.

VP constructed by Gradalis, Inc. (TX, USA), consists of two stem-loop structures with a miR-30a backbone. The bi-shRNA^{furin} DNA as shown in FIG. 1A or 1B uses a single targeted site to induce both mRNA cleavage and sequestration in P-bodies (translational silencing) and/or GW-bodies (repositories). By the use of this proprietary process, the encoding bi-shRNA can accommodate mature shRNA loaded onto more than one type of RNA induced silencing complex (RISC). Also, with the bi-shRNA^{furin} molecular design focusing on a single site potential toxic effects are reduced. Targeting of multiple sites increases chance for a ‘seed sequence’ being induced and leading to off-target effect that could result in increased clinical toxicity. Synthetic complementing and interconnecting oligonucleotides via DNA ligation were used to assemble the two stem-loop double stranded DNA sequences. The 241 base pair DNA constructed with Bam HI sites at both ends was inserted into the Bam HI site of a prior clinically validated plasmid called TAG in which was removed a TGFβ2 antisense DNA sequence and placed the bi shRNA^{furin}-GMCSF DNA sequence. Orientation of the inserted DNA was validated by the appropriate PCR primer pairs designed to screen for the shRNA insert and orientation. Safety profile defined with the prior TAG clinical therapeutic was used to support clinical advancement of VP in experimental cancer management testing under FDA guidance. Vigil is designed with the mammalian promoter cytomegalovirus [CMV] that drives the cassette.

In between the GM-CSF gene (with a stop codon) and furin bi-shRNA there is a 2A ribosomal skip peptide followed by a rabbit poly-A tail. The picornaviral 2A sequence allows the production of two proteins from one open reading frame, by causing ribosomes to skip formation of a peptide bond at the junction of the 2A and downstream sequences. Since the 2A linker has previously been demonstrated to be effective for generating similar expression levels of GM-CSF and anti-TGFβ transcripts with the TAG vaccine and robust activity in product release testing of therapeutic effector components expressed with this plasmid design along with clinical benefit and safety has been observed, the same design for VP was maintained. Transient expression of bi shRNA^{furin} GM-CSF plasmid and diluted expressive cell numbers in patients would not be expected to approach continuous toxic effect of transgenic models.

Gradalis has been clinically testing this plasmid since 2009. Aldevron (ND, USA) and Waisman (WI, USA) have participated in lot manufacturing. This plasmid, which consists of a bi shRNA^{furin} DNA sequence and a GM-CSF DNA sequence (FIG. 1B; SEQ ID NO:3), has been validated for FDA registration trial based on significant cancer patient benefit for use of transfection (via electroporation) into an autologous tumor for vaccine (Vigil) construction in Ewing's sarcoma. Additional benefit has also been suggested in a variety of other cancer types in Phase I and II testing, most notably ovarian cancer. The current active lot of Vigil plasmid was manufactured in 2015 by Waisman. The plasmid concentration per vial is 2.2 mg/ml which provides 770 μg of plasmid. Yearly stability testing has passed all measures of evaluation under US FDA review. Recent double blind randomized control trial involving 25 nationally acclaimed sites unblinded and revealed OS advantage (HR: 0.417, p=0.020) and RFS advantage (HR: 0.459, p=0.007; stratified Cox's proportional Hazard Model).

FIG. 3 shows a lyophilization method 100, wherein the expression vector is in solution 105, which can be rapidly frozen and lyophilized. After lyophilization, the lyophilized powder comprising Vigil becomes a dry powder 110. A dry powder inhaler 115 can aerosolize the dry powder. Those skilled in the art will appreciate that lyophilization is a freeze-drying process in which water is sublimed from the composition after it is frozen. The particular advantage associated with a lyophilization process is that biologicals in an aqueous solution can be dried without elevated temperatures (thereby eliminating the adverse thermal effects), and then stored in a dry state where there are few stability problems. The lyophilized cake containing the expression vectors can be micronized using techniques known in the art to provide 1 μm to 10 μm sized particles.

### Example 2: Proposed Method of Action for Use of Vigil Plasmid in the Treatment of Severe Acute Respiratory Syndrome Coronavirus-2 (SARS-CoV-2) Infection

Severe acute respiratory syndrome coronavirus-2 (SARS-CoV-2) is thought to have naturally evolved from two existing coronavirus strains (L and S) near Wuhan, China. Origin is presumed due to zoonotic transfer: the SARS-CoV-2 genome is 96.2% homologous to the bat RaTG13 coronavirus. From 31 Dec. 2019 to 4 Apr. 2020, 1,133,373 confirmed coronavirus cases have been reported worldwide resulting in 60,375 deaths. Encouragingly, 235,999 patients have shown validated recovery. SARS-CoV-2 is emerging as a potentially greater morbidity and economic threat than the pandemic Spanish flu which infected 500 million people worldwide and resulted in 50 million deaths. The viral reproductive number (RO) of SARS CoV-2 is above two compared with 1.8 for the Spanish flu. Although more highly infectious, SARS-CoV-2 has resulted in far less mortality and is related to primarily elderly patients with medical comorbidities. The current reported mortality rate seems to be holding at approximately 2% worldwide (although age and country related), however, considering shifting denominators, the case fatality rate may be lower; for example, 1.4% of those with laboratory-confirmed disease. Since the pandemic of 1918, influenza has become endemic but with use of vaccines the infection rate has been reduced and the case fatality rate has dropped significantly to 0.1%. The SARS-CoV-2 pandemic, its rate of infectivity, related death, medical system overload and the consequent financial damage (due to disease, fear and quarantine) highlights the urgent need to develop new therapeutics as well as rapid response techniques to combat this and other novel pandemic virus outbreaks in the future.

In particular, patients who recover from SARS-CoV-2 show evidence of an effective immune response to clear the infection and stop viral shedding within approximately 3 weeks. This is particularly important since the virus generally does not persist and viral clearance is achievable, although feasibility of reinfection is unknown given pulmonary site of infectivity and propagation. Efforts can be made to minimize the risk of acute respiratory distress syndrome (ARDS), by enhancing the existing immune response, and slowing the viral propagation process. Repurposing of the Vigil plasmid, which expresses GM-CSF and decreases furin expression, is discussed in this Example. This approach inhibits multiples steps of viral propagation including viral entry, protein assembly and egress while GM-CSF confers cellular immune antiviral and lung protection activity. Pre-clinical and clinical data will be reviewed to highlight the rationale and evidence for combination approach.

### Brief History of Recent Human Coronavirus Infections/Epidemics/Pandemic

Coronavirus was first identified in 1960 in a patient with an upper respiratory tract infection. The virus remained under the radar until 2002 when a patient with severe acute respiratory coronavirus (SARS-CoV) was identified in Guangdong, China. This virus rapidly spread to other hospital patients and staff, then spread globally to 37 countries. Eight hundred of the 8448 individuals diagnosed died. After an initial delay, the measures that were taken limited the degree of dissemination and mortality in comparison to the 1918 pandemic. The next coronavirus outbreak occurred in 2012 beginning in Saudi Arabia in a patient diagnosed with acute pneumonia, the cause of which was identified as Middle East respiratory syndrome coronavirus (MERS-CoV). 2500 cases of MERS-CoV infection were diagnosed in 24 countries of which 800 died before it's resolution in 2014. Thereafter another outbreak in humans occurred in 2015 in South Korea resulting in 186 cases and 36 deaths.

SARS-CoV-2, the virus responsible for COVID-19 is genetically distinct from both the SARS-CoV and MERS-CoV viruses. The first patient was diagnosed in Wuhan, China and subsequently, the virus spread rapidly locally and then escaped regional containment. Over 80,000 cases and 3000 deaths were observed early on. Worldwide travel restrictions and social distancing measures have since been implemented in an attempt to slow the spread and thereby ease the global burden on healthcare workers and facilities. However, worldwide spread continues to occur although regional containment in China and South Korea has been reported, but thus far no well-documented effective therapeutic approach has been found. Although discovery of rapid SARS-CoV-2 infection testing and antibody assessment diagnostics have facilitated identification of hot spot regions. Continued manufacturing of these tests will allow for rapid identification of individuals with SARS-CoV-2 or who have recovered and those who have antibody protection which will facilitate easing social-distancing measures.

### SARS-CoV-2 Morbidity/Mortality Viral Shedding & Immune Response

Preliminary investigation of the first 191 patients in Jinyintan Hospital and Wuhan Pulmonary Hospital revealed that 54/191 (28%) died and 137/191 (72%) were able to be discharged. Analysis of these first 191 patients determined that several factors significantly correlated with risk of death, including age >63 years old, high sequential organ failure assessment score (>1), high D dimer (>1 ng/ml), respiratory rate >24 breaths/min, lymphocyte count >0.0.6×109/l, elevated LDH (median 521 u/l) and elevated IL6 (median 11 μg/ml) as well as comorbidities, hypertension, diabetes, coronary artery disease and COPD. Ninety three percent of the deaths were associated with ARDS and biopsy from one patient showing regions of pulmonary edema with hyaline membrane formation (early-phase ARDS) in one lung and desquamation of pneumocytes and hyaline membrane formation in the other (late-phase ARDS). There is suggestive evidence from the SARS-CoV epidemic that a dysregulated innate immune response and increase of pro-inflammatory cytokines (e.g., IL-1, IL-6 and IFNγ) may contribute to pulmonary pathology. Notably, ARDS is also observed in chimeric antigen receptor CD-19 (CAR-T-CD19) therapy, which targets CD19 antigen and results in rapid induction of IL-6. Given that IL-6 is elevated to greater degree in patients who died from SARS-CoV-2 infection compared with healthy controls, tocilizumab, a monoclonal antibody targeted to IL-6, and used to manage ARDS associated with CAR-T therapy, may be a therapeutic component for those patients with elevated IL-6.

Nasopharyngeal swabs from 79 patients at the First Affiliated Hospital, Nanchang University were obtained to serially assess viral dynamics via PCR-RT. Using the ΔCT method, estimates of viral load were significantly higher by a factor of 60×in severe compared with mild cases and 90% of the latter tested negative by day 10 post onset whereas all the severe cases tested positive beyond that demonstrating both a higher viral load and prolonged shedding time. Using IgM and IgG immunofluorescence (IFA) in 16 patients in Munich, no detectable neutralizing antibody (NA) was detected between days 3 and 6. NA was detected after 2 weeks with limited suggestion of correlation with clinical course. In a recent study of 173 documented (rRT-PCR) cases (161 with serial assessment) of COVID-19 admitted to Shenzhen Third People's Hospital (11 Jan.-9 Feb. 2020), 32 (18.5%) with severe and 141 (81.5%) with mild disease, seroconversion rates in those with serial assessment (ELISA) for IgM and IgG using double-antigen sandwich ELISA for total Ab was 100% (median day 11).

### Severe Acute Respiratory Syndrome Coronavirus-2 (SARS-CoV-2) Dependence on Furin

The β-coronavirus genus is the etiological agent responsible for viral acute respiratory syndromes; the pandemic sarbecoviruses, SARS-CoV and SARS-CoV-2, and merbecovirus, MERS-CoV. SARS-CoV-2 is responsible for the current pandemic of COVID-19 and is distinct from other coronavirus strains. SARS-CoV-2 relies on S1/S2 cleavage at viral entry as compared with SARS-CoV. Following the attachment of the receptor-binding domain (S1) to the ACE2-binding cellular site, the affinity of which is 10- to 20-fold higher than SARS-CoV, the S1 subunit is shed resulting in a stable and accessible fusion domain (S2) subunit. SARS-CoV-2 utilizes the plasma membrane fusion pathway rather than the more immunogenic endosomal membrane fusion pathway, which is used by SARS-CoV. Amino acid sequence differences in the SARS-CoV-2 HR2 region enhances binding affinity between heptad repeat-1 (HR1) and HR2 thereby accelerating viral membrane fusion. The presence of a unique furin cleavage site (RRAR) at the S1/S2 boundary and the furin-like S2′ site located between fusion peptide (FP) and internal fusion peptide (IFP) sites on the S2 subunit may provide a gain-of-function allowing cleavage during viral egress thereby directly or indirectly contributing to increased replication rate, transmission and disease severity. Note that proteolytic cleavage of the S glycoprotein can determine whether the virus can cross species, e.g. from bat. While structurally similar to SARS-CoV-2, the RaTG13/2013 virus lacks a unique peptide PRRA insertion region at the S1/S2 boundary. Further, the S glycoprotein from a MERS-like coronavirus isolated from Ugandan bats can bind to human cells but cannot mediate virus entry unless incubated with trypsin prior to transduction allowing S glycoprotein cleavage and virus entry. These observations suggest that cleavage of the S glycoprotein may be a prerequisite to coronavirus cross-species transmission. A recent publication from Nankai University (Tianjin, China) on SARS-CoV-2 reported that genome sequence analysis revealed a section of genes that was not present in SARS-CoV that had a cleavage site similar to HIV and Ebola which carry viral proteins necessary for fusogenic activity of viral species to the human cell membrane. To be activated, the viral fusogenic surface glycoprotein has to be cleaved by furin. As mentioned, viruses contain surface glycoproteins which when cleaved by furin or other proprotein convertases (PC) are activated and viral propagation is achieved (i.e., avian influenza, HIV, Ebola, Marburg and measles viruses). Another PC necessary for viral entry is the transmembrane serine protease TMPRSS2, which is known to contribute to efficient SARS-CoV cell entry and in vitro data has been produced showing that SARS-CoV-2 also uses TMPRSS2 priming. However, further assessment of furin cleavage in vivo is appropriate given fusion-mediated cell entry of SARS-CoV-1 rather than SARS-CoV via endocytosis, the presence of a unique furin cleavage site (RRAR) at the S1/S2 boundary and the furin-like S2′ site in SARS-CoV-2 and the combination of cell membrane entry fusion and differences in the SARS-CoV-1 HR1 domain, which may contribute to the typical syncytium growth pattern in infected cells rarely reported in SARS-CoV. Inhibition of furin may be a therapeutic approach that has efficacy in SARS-CoV-2 and other viruses that contain a furin cleavage domain. Another immunotherapeutic intervention would be to increase the pulmonary expression of GM-CSF, which, in vivo, redirects macrophages from an M1 state of activation to an M2 activation state and enhances expression of anti-inflammatory mediators and perhaps allow more time for patients to mount an effective immune response against SARS-CoV-2.

In addition to interfering in viral dynamics, a therapy-targeting host proteases rather than a viral epitope could also reduce the development of vaccine resistance due to mutation of nonessential viral-targeted antigens. For both reasons, furin is an attractive therapeutic target. It is highly conserved and genomically unrelated to viral replicative functions and antigenic drift. It is not known how effective vaccination will be with SARS-CoV-2 given the low titers of NA in patients with COVID-19 and antigenic drift characteristic of human host RNA viruses. Vaccination for influenza virus is only effective in 60% of individuals due to rapid antigenic evolution.

### Furin

Furin, was first described in 1986 and is the product of the fur gene. It is an evolutionarily conserved family member of the proprotein convertases which contain a subtilisin-like protease domain and was the first proprotein convertase (PC) to be identified in humans. Furin is a type I transmembrane protein that is ubiquitously expressed in vertebrates and invertebrates. It is localized to the Golgi and trans-Golgi network where it cleaves multiple proteins and is also located on the outer membrane where pathogens utilize it to cleave glycoproteins, a step essential for entry into host cells. It can be secreted as a soluble, truncated active enzyme. The correct folding of furins catalytic domain relies on the inhibitory function of the N-terminal 83-amino acid propeptide. To gain its enzymatic activity, the inhibitory propeptide is removed during transport from the endoplasmic reticulum to the trans-Golgi network. In order to be released into the extracellular space, the membrane localization is cleaved at the C-terminus. Due to furin's ubiquitous expression and localization it is able to process a large amount and variety of proteins including growth factors, cytokines, hormones, adhesion proteins, collagens, membrane proteins, receptors as well as other classes. Furin cleavage can also inactivate other proteins. Its cleavage consensus sequence is Arg-Xaa-(Lys/Arg)-Arg↓-Xaa.

Many viral pathogens including, coronavirus, flavivirus, pneumovirus, avian influenza, influenza A and HIV, utilize furin-mediated membrane glycoprotein cleavage facilitate viral entry and, for certain viruses, egress from target host cells. HIV-1 utilizes furin to cleave the viral membrane protein (Env) gp160 into gp120 and gp-41 prior to mature virion assembly. Conversely, flavivirus rely on furin cleavage after formation of packaged virions. SARS-CoV-2, as noted above, is cleaved at two sites, S1/S2 furin cleavage site (PRRAR↓SV) and a furin-like S2′ cleavage site (KR↓SF).

### Viral Response to Furin Protein Inhibitors

RNA viruses such a SARS-CoV-2 have several critical functions dependent upon protease activity. Consequently, modulation of protease activity may provide therapeutic function in SARS-CoV-2 in a variety of other RNA viruses. Furin is a particularly promising opportunity for therapeutic intervention. As previously described, it cleaves and activates numerous mammalian, viral and bacterial substrates. Optimized preclinical therapeutic performance of several peptidomimetic furin inhibitors and demonstrated ‘in vitro’ significant inhibition of highly pathogenic H7N1 influenza virus propagation.

Although mechanisms have evolved enabling RNA viruses to invade host cells, host defense mechanisms have also evolved. Innate and adaptive immune responses have been shown to target viral antigens. Additionally, targets critical to viral entry, protein assembly and egress are also of high therapeutic value. These are ‘virus dependency factors’. Various host proteins such as IFI16 and SAMHD1 have been shown to inhibit both RNA and DNA viral gene expression and replication, respectively. Furin is critical for viral membrane fusion, protein assembly and propagation, particularly as related to SARS-CoV-2.

Multiple furin inhibitors have been developed and tested in vitro and in animal models. Initial targets were peptide and protein inhibitors which target active sites and competitively inhibit binding sites. As example, two IFNχ-inducible GTPases, guanylate-binding proteins 2 and 5 (GBP2 and GBP5), with inhibitory furin activity have demonstrated cleavage inhibition of the HIV Env precursor gp160 and reduced HIV virion infectivity. Control of furin expression with protease activated receptor 1 (PAR1), impacts downstream furin function and processing of human metapneumovirus F protein in HIV. Associated neurocognitive disorders also provides evidence of resistance mechanisms that can occur while inhibiting spread of HIV-1. Another example, α-1 antitrypsin Portland (α1-PDX) inhibits both PC5K5 and furin. α1-PDX has been shown to inhibit processing of HIV-1 Env and measles virus F. Moreover, peptides involving the cleavage site of influenza A virus hemagglutinin compete for furin activity. Activation of MMP9 is also inhibited by autoinhibitory propeptide of furin. These data support therapeutic development involving furin inhibition against SARS-CoV-2.

Interestingly, corneal damage in mice related to Pseudomonas aeruginosa has been shown to be reduced by non-D-arginine (D9R) and other furin inhibitors. Nonpeptidic furin inhibitors have also demonstrated antifurin activity in the nanomolar dose range. 2,5-dideoxystreptamine shows unusual furin inhibiting activity whereby a complex is formed with furin involving two molecules with separate functions, which interfere with the catalytic triad conformation and binding to an adjacent peptide stretch to inhibit furin activity.

Toxic effects related to furin inhibitors have not been observed outside of embryonic models. A study of furin-deficient mice demonstrated a critical role of furin during embryogenesis in which knock-out of the fur gene led to death by day 11 due to the failure of ventral closure and embryonic turning. Therefore, furin inhibition should be limited to the non-pregnant population. Liver-specific interferon-inducible furin knock-out mice have not demonstrated adverse effects outside of embryogenesis implying that other proprotein convertases may compensate for furin deficiency given overlapping activity. Targeting furin, a host enzyme, also avoids the emergence of resistance due to viral antigenic drift as described earlier as furin genome is highly conserved and maintains a stable genomic structure, while SARS-CoV-2 target sites undergo mutational changes throughout the viral life span and pandemic period. Furin inhibitors also function as mentioned previously via knockdown at the RNA level [i.e., Regnase-1 (ZC3H12A), Roquin (RC3H1)]. A concern, however, with modulation of Regnase-1 and Roquin is that both agents will most likely result in off-target effects as these products both degrade off target mRNA. The results outlined and safety profile support potential role of furin inhibitors within a pandemic and possibly even within the anti-terrorist government protection ‘tool box’.

### GM-CSF Antiviral Activity

Similar to SARS-CoV-2, alveolar epithelial cells are the primary target of influenza virus (IV) and are the first site of entry and support for viral propagation and replication. Proinflammatory immune response is rapidly initiated toward viral cytopathogenic effect which leads to alveolar epithelial cell (AEC) apoptosis. However, when infection persists and viral propagation continues leading to intensified inflammatory response, capillary and alveolar leakage occurs, followed by severe hypoxemia and eventually ARDS which requires hospitalized management, oxygen support and often ventilation assistance. Clearance of the viral pathogens from the lung by immune effector cells and the initiation of epithelial repair processes including expansion of local epithelial progenitor cells to begin resealing of the epithelial layer are critical for medical recovery and prevention of hospitalization, oxygen and ventilation support in IV-induced lung injury. The majority of mortality in relation to SARS-CoV-2 infection has been related to ARDS leading to hospitalization and ventilation support which is testing our medical capacity. However, the inflammatory immune response against the virus needs to be balanced between the elimination of virus and toxic effect of immune-mediated pulmonary injury in order to limit damage to the respiratory tract and alveolar cells which prevent ARDS. Mononuclear effector cells (macrophages, dendritic cells, CD8+, neutrophils and lymphocytes) carry the bulk of the load in IV clearance and ‘balanced’ immune response against IV. Similar activity demonstrated with IV is important for clearance of SARS-CoV-2. GM-CSF has been shown to promote proliferation, differentiation and immune activation of monocytes, granulocytes, macrophages. GM-CSF in the lungs is mainly expressed by AEC type II cells and is a first cytokine responder in protection of the lung environment, AEC survival and function, and is a positive prognostic factor in clearance of IV infection. Expressed GM-CSF in pulmonary secretions can potentially be used as an indication in bronchial lavage samples of early response and resistance thereby affecting medial need involving O₂ support. Other cell types produce GM-CSF, but AECs have been shown to upregulate GM-CSF in the distal lung parenchyma upon IV infection, and then produce high levels of GM-CSF in the alveolar surrounding secretions. AEC GM-CSF secretion with IV infection resolution appears to be further mediated via HGF/c-Met and TGF-α/EGFR signaling.

Relationship of GM-CSF to immune response activation against cancer and viral infection is well described. GM-CSF also regulates the differentiation, proliferation and activation of alveolar macrophages. In vitro studies indicate that GM-CSF causes rapid proliferation of alveolar type II epithelial cells thereby serving in repair and barrier protection of the respiratory epithelium at early stages of acute inflammation. It is also known that GM-CSF expression from alveolar type II epithelial cells facilitates surfactant homeostasis further enhancing protection of viral induced pathology. GM-CSF also enhances the antiviral responses of alveolar macrophages. Indeed, elevated levels of GM-CSF may elicit a biphasic M1↔M2 response pattern. Although a number of studies show that GM-CSF and type I interferon act together to modulate macrophage polarization toward the M1 state of activation, recent in vivo studies conclude the opposite. GM-CSF enhances viral clearance through expression of scavenger receptors, SR-A and MARCO. These two receptors aid in viral clearance through activation of receptors TLR-3, TLR-9, NOD-2 and NALP-3. GM-CSF enhances mucosal immune responses and the effectiveness of DNA vaccines. Recombinant human GM-CSF has been delivered to the lung and conferred resistance to IV infection. Transgenic mice that constitutively expressed human GM-CSF exposed to IV, were able to mount and effective antiviral response that resulted in increased numbers of human alveolar macrophages. GM-CSF overexpression has been shown after IV virus infection in a GM-CSF transgene mouse model prevents mortality. Protective effects of GM-CSF against IV-A pneumonia have been seen in mice with constitutive and inducible GM-CSF expression models in alveolar type II epithelial cell transgenic mice with GM−/− and GM+/+ pulmonary specific promoters (SFTPC, SCGB1A1), respectively. This model was able to show GM-CSF enhancement of alveolar cell activity as indicated by increased expression of SP-R210 and CD11c expressive mononuclear cells. In mice lacking SR-A and MARCO, two receptors regulated by GM-CSF, MARCO was shown to increase expression of SP-R210 on alveolar macrophages and decrease resistance to IV. However, although continuous SP-C-GM+/+ transgenic mice resisted early mortality from IV, concern was raised to continuous high GM-CSF exposure over prolonged time. Late assessment of lung tissue sections revealed the histological features of degenerative desquamative interstitial pneumonia at day 29. Degeneration of alveolar structure and large spaces containing desquamated cells characterized the lungs of high GM-CSF exposed mice. The results indicate that excessively high levels of GM-CSF impair appropriate tissue healing resulting in development of interstitial lung disease secondary to IV pneumonia and provide guidance for early large animal assessment and Phase I monitoring of patient safety. However, results support that the conditional GM-CSF expressive mice do well and have long-term survival advantage to IV infection and have significant advantage over untreated controls. Expression of GM-CSF either through transgenic or pulmonary delivery conferred survival advantage to influenza virus compared with WT mice that did not survive infection. When alveolar phagocytes were depleted, the protective effect also diminished suggesting that these cells are necessary to induce the innate immune response.

As described, infection with SARS-CoV-2 can progress to rapid induction of viral pneumonia and ARDS resulting in fatal outcome. AECs play a critical role in orchestrating the pulmonary antiviral host response. However, with early IV and SARS-CoV-2 infection AEC's release GM-CSF. GM-CSF heightens immune function of alveolar cells which leads to improved epithelial repair processes. During IV infection, AEC-derived GMCSF also enhances a lung-protective mechanism. Similar results are seen with local rhGM-CSF application. This early use and/or enhancement of immune function and alveolar protection with elevated GM-CSF expression appears to overwhelmingly benefit clinical response. However, GM-CSF expression late in the inflammatory lung response is less well characterized. Although using recombinant GM-CSF (leukine, Bayer HealthCare Pharmaceuticals, WA, USA) and an Aeroneb Solo nebulizer to administer leucine (125 μg/dose) demonstrated significant clinical benefit in four of six patients with ARDS related to infectious pneumonia (including two with H1N1 virus). Immune function enhancement was also shown in the leucine treated patients compared with untreated ARDS patients in analysis of pulmonary immune response, which is similar to preclinical evidence (in vitro and animal models). GM-CSF treated patients demonstrated alveolar cell protection, enhanced alveolar cell activity toward viral and other infectious clearance and shift to M1 response as assessed by increased alveolar CD80+ cells and CD206 drop. These results support enhancement of GM-CSF expression even late in pulmonary inflammatory response to viral infection may be of benefit which suggest therapy benefit in late stage ARDS patients. Safe administration of GM-CSF via inhalation therapy in 19 patients with autoimmune pulmonary alveolar proteinosis was also demonstrated to show benefit. Elevated IL-17 in bronchial alveolar lavage fluid was shown as a GM-CSF induced cytokine and may serve as a biomarker associated with benefit.

Worsening IV infection and response in GM-CSF deficient mice has found to be due to impaired IV clearance by macrophages. It has been demonstrated that the Fcγ receptor (FcγR)-mediated opsonophagocytosis of invaded pathogens by alveolar macrophages is related to GM-CSF. T-cell-produced interferon γ (IFN γ) also effects alveolar macrophage FcγR expression which in turn stimulate production of IFN γ and other cytokines such as IL-18 and IL-12 supporting involvement of both innate and adaptive immunity turn on. Elevated alveolar GM-CSF level in transgenic mice also improves resistance of alveolar cells in association with IV infection. GM-CSF has also been shown to be an important stimulator of CD8+T lymphocytes and further enhances their role to activate DC priming in lymphoid tissue, thereby providing a positive feedback in further stimulation of CD8+ T cell expansion. GM-CSF has been shown to be critically important for induction of CD8+ T-cell immunity GM-CSF has also been shown to promote B-cell maturation and production of IV specific antibodies. During IV pneumonia, extensive additional in vivo data support the role of GM-CSF as a lung barrier-protectant and positive immune response factor. AEC-expressed GM-CSF directly benefits the injured epithelium and is important in enhancing epithelial proliferation in the setting of hypoxic lung injury via repair of barrier function, reduction of capillary leak and return of tissue to homeostasis.

The data discussed above regarding targeting furin and increasing GM-CSF expression warrants further investigation to target SARS-CoV-2 infection. Vigil, which combines bifunctional shRNA targeting furin and incorporating a GM-CSF DNA sequence in a plasmid delivery vehicle (pbi-shRNA^{furin}-GM-CSF) has been described as the most advanced anti-furin technology in clinical testing. Vigil is an autologous tumor cell vaccine, with dual function that knocks down furin expression as seen by decreased expression of downstream proteins TGFβ1/2 and expresses GM-CSF. It has demonstrated clinical success in several cancer populations but especially Ewing's sarcoma and ovarian cancer. It has a demonstrated safety profile with no evidence of grade 3 product related toxicity effect following 1406 doses in 233 cancer patients.

The potential efficacy and use of Vigil for COVID-19 is an example of the rational repurposing of drugs from indicated to nonprimary target disease alternatives. Such an approach could accelerate the clinical development process particularly urgent given the current COVID-19 pandemic.

### Anti Furin Therapeutic: GM-CSF Bi-shRNA^{furin} Plasmid (VP)

VP constructed by Gradalis, Inc. (TX, USA), consists of two stem-loop structures with miR-30a backbone. The bi-shRNA^{furin} DNA as shown in FIG. 1A or FIG. 1B uses a single targeted site to induce both mRNA cleavage and sequestration in P-bodies (translational silencing) and/or GW-bodies (repositories). By the use of this proprietary process, the encoding bi-shRNA can accommodate mature shRNA loaded onto more than one type of RNA induced silencing complex (RISC). Also, with the bi-shRNA^{furin} molecular design focusing on a single site potential toxic effects are reduced. Targeting of multiple sites increases chance for a ‘seed sequence’ being induced and leading to off-target effect that could result in increased clinical toxicity. Synthetic complementing and interconnecting oligonucleotides via DNA ligation were used to assemble the two stem-loop double stranded DNA sequences. The 241 base pair DNA constructed with Bam HI sites at both ends was inserted into the Bam HI site of a prior clinically validated plasmid called TAG in which was removed a TGFβ2 antisense DNA sequence and placed the bi-shRNA^{furin}-GMCSF DNA sequence. Orientation of the inserted DNA was validated by the appropriate PCR primer pairs designed to screen for the shRNA insert and orientation. Safety profile defined with the prior TAG clinical therapeutic was used to support clinical advancement of VP in experimental cancer management testing under FDA guidance. Vigil is designed with the mammalian promoter cytomegalovirus [CMV] that drives the cassette. In between the GM-CSF gene (with a stop codon) and furin bi-shRNA there is a 2A ribosomal skip peptide followed by a rabbit poly-A tail. The picornaviral 2A sequence allows the production of two proteins from one open reading frame, by causing ribosomes to skip formation of a peptide bond at the junction of the 2A and downstream sequences. Since the 2A linker has previously been demonstrated to be effective for generating similar expression levels of GM-CSF and anti-TGFβ transcripts with the TAG vaccine and robust activity in product release testing of therapeutic effector components expressed with this plasmid design along with clinical benefit and safety has been observed, the same design for VP was maintained. Transient expression of bi-shRNA^{furin}-GM-CSF plasmid and diluted expressive cell numbers in patients would not be expected to approach continuous toxic effect of transgenic models.

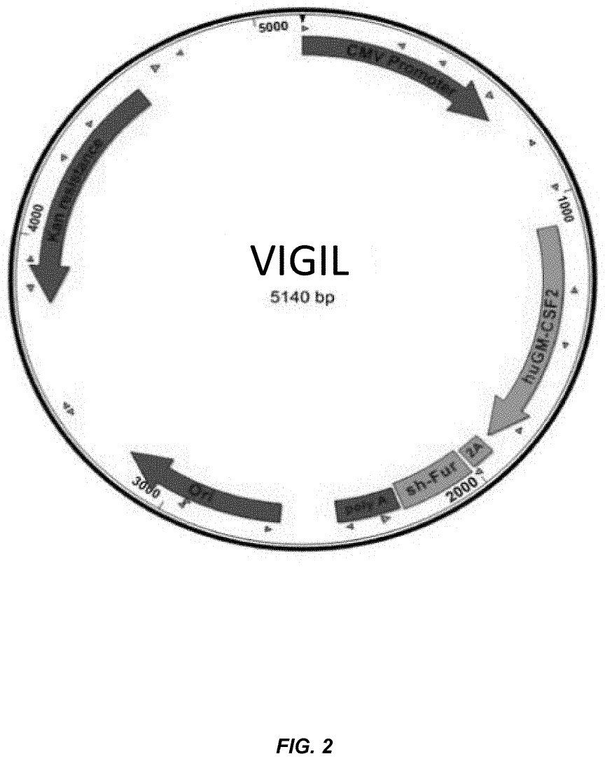

Gradalis has been clinically testing this plasmid since 2009. Aldevron (ND, USA) and Waisman (WI, USA) have participated in lot manufacturing. This plasmid, which consists of a bi-shRNA^{furin} DNA sequence and a GM-CSF DNA sequence (FIG. 2), has been validated for FDA registration trial based on significant cancer patient benefit for use of transfection (via electroporation) into an autologous tumor for vaccine (Vigil) construction in Ewing's sarcoma. Additional benefit has also been suggested in a variety of other cancer types in Phase I and II testing, most notably ovarian cancer. The current active lot of Vigil plasmid was manufactured in 2015 by Waisman. The plasmid concentration per vial is 2.2 mg/ml which provides 770 μg of plasmid. Yearly stability testing has passed all measures of evaluation under US FDA review. Recent double blind randomized control trial involving 25 nationally acclaimed sites unblinded and revealed OS advantage (HR: 0.417, p=0.020) and RFS advantage (HR: 0.459, p=0.007; stratified Cox's proportional Hazard Model).

### Aerosolized Therapeutics for Viral Pneumonia

Viral pneumonia, particularly in elderly or immune compromised patients, can be associated with devastating medical consequence. Pulmonary delivery via aerosolized systems are simple, nonexpressive, noninvasive and allow for pain-free access of therapeutic and minimization of possible systemic side effects. Aerosols have been shown to deliver plasmid DNA droplets with size ranging from 1 and 5 μm, which are able to disperse to the bronchial and alveolar epithelial cells. This enables pDNA entry and maximizes subsequent gene expression. The use of a SAW liquid nebulization device for the generation of aerosolized pDNA with suitable size and stability characteristics to facilitate effective pulmonary delivery particularly for IV vaccination has been demonstrated. In vivo studies have shown successful pDNA delivery in both small and large animals. SAW nebulization used to deliver a plasmid vaccine demonstrated expression of protective anti-hemagglutinin (HA) antibodies. Anti-HA antibody titers detected were comparable to vaccination outcomes of other similar pDNA influenza vaccines not using a nebulizer. These results support use of naked pDNA for effective delivery via pulmonary distribution while also demonstrating product stability and function. Following pDNA vaccination in rats, revealed higher serum hemagglutination inhibition (HAI) titers which were identified as protective according to WHO standards. However, at this time, the SAW nebulizer approach has not demonstrated scale up capability for use in a pandemic event.

Aerosolized ribavirin however has demonstrated large volume capacity and adequate aerosolized delivery and clinical benefit including use in morbid condition patients. Ribavirin is indicated therapy for severe RSV infection in children. The conventional continuous treatment of 60 mg of ribavirin/ml for 18 h was found to be effective. Aerosolized ribavirin (administered 20 mg/ml for 2 h three times daily) has also been effective in cancer patients with RSV infection. Ribavirin inhalation method at intermittent high doses (60 mg/ml) over the same schedule in immune suppressed children with RSF infection was also well tolerated. Moreover, results demonstrated similar improved clinical response compared with standard therapy. There was also less adverse exposure to healthcare workers. Parainfluenza virus is associated with potentially serious complications in high morbidity patients (i.e., heart-lung transplant, allograft rejection, bronchiolitis obliterans. Inhaled ribavirin in this population was associated with clinical improvement. Aerosolized ribavirin (60 mg/ml) was also effective against IV-A and B infections in mice. Recently, aerosolized ribavirin (100 mg/ml) was shown to be effective in mice infected with lethal IV-A H3N2 virus, and resulted in >0% survival when given early (within 24-48 h) after infection. Aerosolized ribavirin treatment has been used with success against metapneumovirus pneumonia. Moreover, in treatment of pneumotropic human adenovirus, aerosolized ribavirin demonstrated greater benefit over intravenous ribavirin likely related to the more robust drug concentration achieved in the alveoli with aerosolized product compared with intravenous ribavirin therapy. Additionally, the aerosolized delivery did not appear to lead to cytotoxic effect. S-FLU immunization provides a broad cell-mediated immune response to conserved viral antigens. Data reveal that immunization with S-FLU-expressing H1 HA (H1 S-FLU) DNA reduces the viral load in lungs after aerosolized challenge with the closely matched pdmH1N1 virus strain. The reduction of viral load was shown to be optimal using aerosol administration when compared with intravenous S-FLU. However, viral neutralizing Ab was not observed in S-FLU-immunized pigs, and the reduction of viral load in the H1 are group correlated with the presence of IFNg-producing CD8 or CD4/CD8 double-positive cells in the bronchoalveolar lavage suggest adequate product delivery. These data provide proof of principle that S-FLU DNA can be efficiently delivered by aerosol to a large animal, supporting possible use of a nebulizer device as a method of immunizing patients. Aerosolized delivery may be further optimized with use of lipid-DNA complexes. Others have also shown successful aerosol delivery of measles vaccine in humans and/or exosome/viral delivery.

The challenge to this approach is how to introduce plasmid DNA into the lungs without loss or damage to the plasmid. Plasmid DNA is highly prone to shearing, therefore methods with low shear forces are necessary for effective delivery of the supercoiled DNA. Both nebulizers and dry powder inhalers use low amounts of shear forces. Nebulizers however use aerosol droplets to deliver particles into the lungs, which may not be an effective method to deliver plasmid DNA, as DNA degrades while in solution if not stored appropriately. Additionally, nebulizers limit the concentration of product that can be delivered due to solubility. Dry powder inhalers are not limited by solubility and plasmid DNA would not need to be stored in solution. This method also reduces shear stress and thermal degradation which results in a high concentration of quality plasmid delivered directly into the lungs.

### Conclusion

Accumulating knowledge of intracellular viral processing, molecular biology, viral dynamics, host immune mechanisms and immunokinetics will allow for the development of tools and methods to protect lung function, delay or prevent ARDS, enhance anti-viral resistance and institute prophylactic measures. The unique role of furin and the demonstration of robust viral clearance in all patients who survive SARS-CoV-2 infection supply the rationale and support for repurposing Vigil for treatment of patients with COVID-19. Knockdown of furin with Vigil would target multiple steps of viral propagation, including viral, entry, protein assembly and egress. Expression of GM-CSF would provide further therapeutic benefit, enhancing the immune response and AEC protection. The data, limited as it is, showing no obvious correlation between seroconversion and viral clearance, gives additional support to a multifunctional therapeutic approach to COVID-19, in other words, combining inhibition of a protease critical to viral entry and cell to cell transmission with an immune response modulator. Vigil is already involved in FDA characterization with a known product safety profile. Further testing will be necessary, including in vitro activity assessment against SARS-CoV-2 and large animal safety.

### Example 3: Vigil Plasmid (VP), a Dual Bi-shRNA^{furin}-GMCSF Construct from Cancer to SARS-CoV-2

SARS-CoV-2 genome reveals a unique furin cleavage site change at S1/S2 junction and furin-like S2′ cleavage site which promotes membrane fusogenic pathway entry and exit to human host cells. Clinical testing of VP, used in an autologous tumor vaccine (Vigil), demonstrates >90% knockdown of TGFβ, a downstream furin protease product, elevation of GMCSF and safety and benefit in solid tumor cancer patients.

Lyophilized (Lyo) VP blended with GFP was stabilized with w/v Trehalose to ≤5 micron particle size for deep lung entry. Significant GFP expression was demonstrated (Nexcelom Cellometer) and restriction enzyme mapping confirmed molecular structure. GMCSF and TGFβ expression (Protein Simple ELLA cytokine production) in CCL247 and RDES cell lines validated for performance of FDA defined cancer clinical product release assays was done. Function was determined for both electroporation (BioRad) and lipid-based reagent [Lipofectamine (Lipo) 3000].

Transfection efficiency and cytokine expression of non Lyo and Lyo VP with and without electroporation (Zap) are shown in TABLE 2.

TABLE 2
Transfection efficiency and cytokine expression.

| | Cell Line | | | | | |
|---|---|---|---|---|---|---|
| | CCL247 | | | RDES | | |
| | Time Point | | | | | |
| | Day 2 | | | Day 3 | | |
| | Treatment Condition | | | | | |
| | Zap + | Lipo + | Lipo + | Zap + | Lipo + | Lipo + |
| | Frozen | Frozen | Lyo DNA | Frozen | Frozen | Lyo DNA |
| | DNA | DNA | (5% Tre) | DNA | DNA | (5% Tre) |
| GFP (Transfection | 65% | 50% | 70% | 69% | 50% | 52% |
| efficiency) | | | | | | |
| GMCSF (pg/mL) | 1.85 e6 | 3.01 e6 | 3.82 e6 | 3.95 e6 | 4.84 e6 | 5.08 e6 |
| TGFβ1 (% Knockdown) | 54% | 23% | 35% | 59% | 30% | 41% |

### Example 4: Determination of Antiviral Activity of Bi-shRNA^{furin}-GMCSF Vigil Plasmid (VP)

Cell culture, transfection and virus infection: Vero E6 cells were purchased from the American Type Culture Collection (Manassas, Va., USA). SK-N-SH cells was kindly provided by Dr. Richard Wozniak (University of Alberta, Canada). SK-N-SH and Vero E6 cells were cultured in Dulbecco's modified Eagle's medium (DMEM; Gibco; Waltham, Mass., USA) supplemented with 10% heat-inactivated fetal bovine serum (FBS; Gibco; Waltham, Mass., USA), 100 U/mL penicillin and streptomycin, 4.5 g/l D-glucose, 2 mM glutamine, 110 mg/l sodium pyruvate at 37° C. in a 5% CO₂ atmosphere. Contemporary isolate 72B/CA/CALG D614G of SARS-CoV-2 was used. Virus manipulations were performed under biosafety CL-3 containment procedures. SK-N-SH cells were transfected with plasmids (UMVC plasmid and Vigil plasmid) using Lipofectamine 2000 (Invitrogen) as described in the detailed experiment protocol below. UMVC plasmid was purchased from Aldevron. For the UMVC plasmid, starting with the commercial UMVC vector, we cloned in GM-CSF, 2a linker, and TGFB2 antisense to make TAG.

At 24 hrs post-transfection, cells were infected with SARS-CoV2/72B/CA/CALG D614G with MOI=0.1 for 24 hours and 48 hours. Supernatants containing viruses at each time pointe were collected, filtered, aliquoted and kept at −80° C. Virus titers were determined using plaque assay. Cells were washed twice with PBS and lysed in RA1 buffer, provided in NucleoSpin RNA Kit (Machery-Nagel). Total RNA was then isolated following the manufacturer's protocol. A day-by-day protocol is provided below:

Day 0: Seed SK-N-SH cells in 6-well plate (400,000 cells/well, gave about 70-80% cell confluence at transfection) (Day 0).

Day 1: Next morning, transfect plasmid UMVC and VP using Lipofectamine 2000 (Invitrogen) in 200 μL Opti-MEM (Gibco) by diluting 3 μL of Lipofectamine 2000 reagent in 100 μL Opti-MEM medium, diluting 3 μg of DNA in 100 μL Opti-MEM medium, and adding diluted DNA into diluted Lipofectamine 2000 reagent (1:1 ratio). Incubate for 15 minutes and then add DNA-lipid complex to cells. No DNA transfection or the UMVC plasmid was used as a negative control.

Day 2: 24 hrs post-transfection, infect with SARS-CoV2/72B/CA/CALG D614G with MOI=0.1 for 1 hr, remove viruses, wash twice with PBS, and replace with fresh medium. Let the infection go for 24 hours and 48 hours.

Days 3 and 4: At 24 hrs and 48 hrs post-infection, supernatants containing viruses were cleared of cells and debris, filtered, aliquoted and kept at −80° C. Virus titers were determined using plaque assay. Cells were washed twice with PBS and lysed in RA1 buffer, provided in NucleoSpin RNA Kit (Machery-Nagel). Total RNA was then isolated following the manufacturer's protocol.

Quantitative Real-Time PCR (qRT-PCR): For RNA analysis, total RNA from SK-N-SH cells was extracted using the NucleoSpin RNA Kit (Machery Nagel; Bethlehem, Pa., USA) following the manufacturer's protocol. 0.5 to 1 μg of total RNA was reverse transcribed with random primers (Invitrogen; Carlsbad, Calif., USA) and the Improm-II reverse transcriptase system (Promega; Madison, Wis.) at 42° C. for 1.5 h according to the manufacturer's protocol. The resulting cDNAs were mixed with the appropriate primers (Integrated DNA Technologies; Coralville, Iowa) and PerfeCTa SYBR Green SuperMix Low 6-Carboxy-X-Rhodamine (ROX) (Quanta Biosciences; Beverly, Mass.) and then amplified for 40 cycles (30 s at 94° C., 40 s at 55° C. and 20 s at 68° C.) in a CFX96 Touch™ Real-Time PCR Detection System (Bio-Rad). Gene expression (fold change) was calculated using the 2(−ΔΔCT) method using human β-actin messenger RNA transcript as the internal control. The following forward and reverse primer pairs were used for PCR: β-actin 5′-CACCATTGGCAATGAGCGGTTC-3′ (SEQ ID NO:10) and 5′-AGGTCTTTGCGGATGTCCACGT-3′ (SEQ ID NO:11), SARS-CoV-2 spike 5′-CCTACTAAATTAAATGATCTCTGCTTTACT-3′ (SEQ ID NO:12) and 5′-CAAGCTATAACGCAGCCTGTA-3′ (SEQ ID NO:13), furin 5′-GCCACATGACTACTCCGCAGAT-3′ (SEQ ID NO:14) and 5′-TACGAGGGTGAACTTGGTCAGC-3′ (SEQ ID NO:15).

Plaque Assay:
a. Vero E6 cells were plated in a 24-well plate at 1.0×10⁵ cells per well and incubated overnight at 37° C. Plate enough wells to test each dilution in duplicate (starting from 10⁻¹ to 10⁻⁶; 10-fold dilutions).
b. Dilute samples to be titered in DMEM media in 96-well plates. Make a 10-fold dilution series, providing enough volume to add 100 μL per well in 24-well plate.
c. Remove existing cell culture media from 24-well plates and add 100 μL media per well. Add 100 μL of each dilution to one well of a 24-well plate.
d. Incubate 24-well plates at 37° C. in 5% CO₂ for 1 h, rocking plates every 15 min to prevent cells from drying out.
e. Meanwhile, Place 1×MEM+2% FBS with 0.75% methylcellulose plaque media in 37° C. incubator while plates are incubating to decrease viscosity of the solution.
f. After 1 h incubation, remove the virus and add 1 mL plaque media to each well of the 24-well plates. Incubate plates at 37° C. in 5% CO₂ for 3 days.
g. On day 3, gently remove methylcellulose overlays and fix cells by adding 1 mL of 4% paraformaldehyde (PFA) in PBS to each well. Incubate at room temperature for 30 min.
h. Remove 4% PFA into an appropriate hazardous waste container. Wash wells with dH₂O and then add 1 mL of 0.05% (w/v) crystal violet in 20% methanol to each well. Incubate for 30 min. Remove crystal violet with a pipette and wash with dH₂O until excess crystal violet is removed, and plaques are easily visualized.
i. Count the plaques at the dilution in which there are 5-30 plaques. Calculate titer in PFU/mL using the following formula: Titer (PFU/mL)=number of plaques counted×10\hat{ }^{dilution counted}×10 (to get to mL because 100 μl of diluted sample were added).

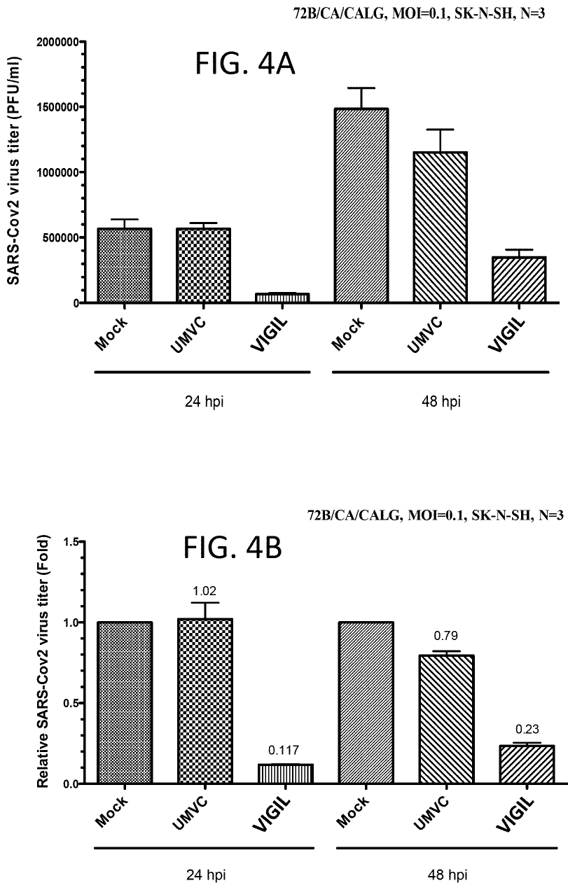

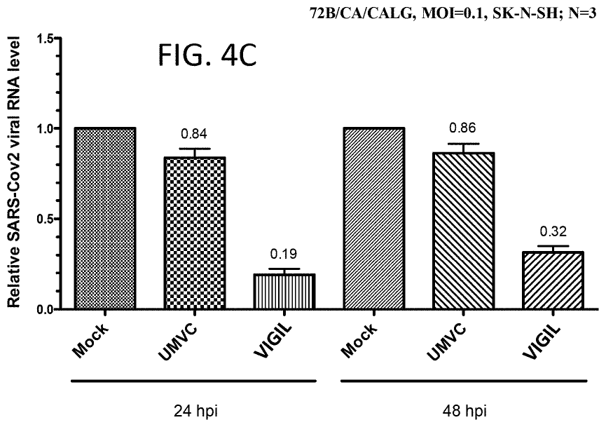

FIGS. 4A-4C show that knockdown of furin reduces SARS-COV-2 viral RNA using contemporary 72B/CA/CALG strain (0.1 MOI) w/SK-N-SH cells, N=3.

### Example 5: Lyophilized Plasmid

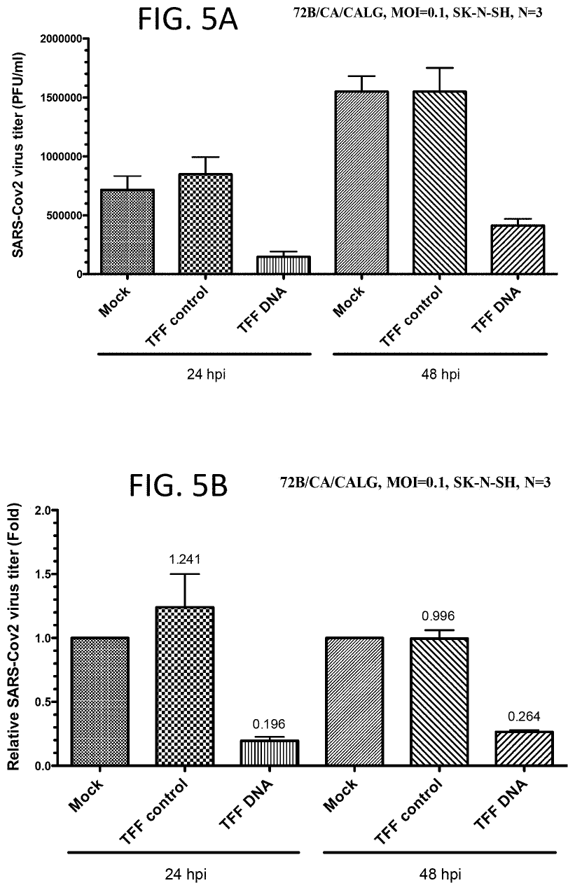

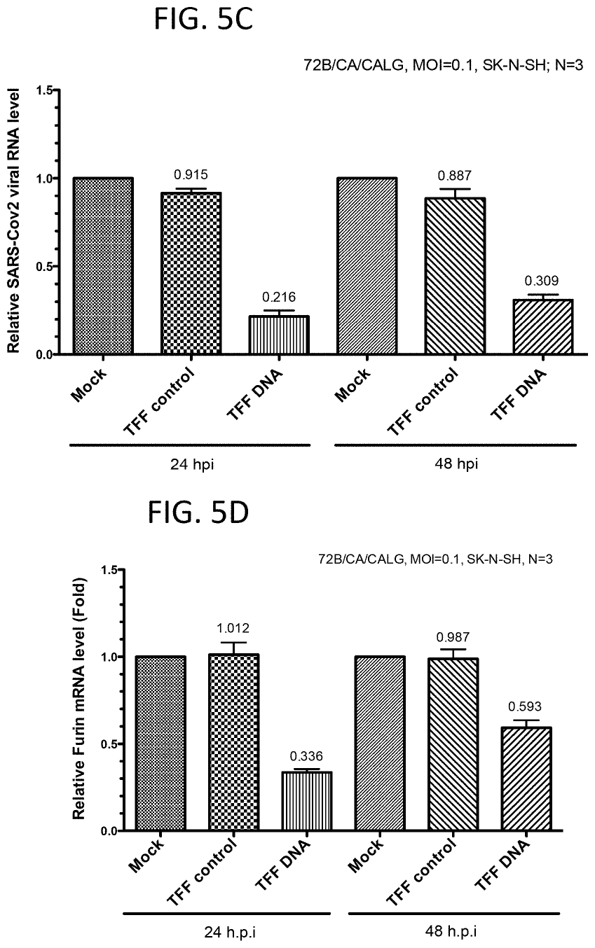

The antiviral effects of lyophilized plasmid was also tested. As shown in FIGS. 5A-5D, lyophilized plasmid from thin film freezing (TFF) performed similarly to frozen/thawed Vigil plasmid, which demonstrated that the lyophilized plasmid had antiviral effects and that the plasmid did not denature and remained stable.

FIGS. 5A-B show that knockdown of furin reduces SARS-COV2 virus titer. In addition, FIG. 5C shows that knockdown of furin reduces SARS-COV2 replication. FIG. 5D shows the furin knockdown efficiency by TFF DNA.

### Embodiments

Embodiment 1. A method of treating a respiratory condition caused by a virus in an individual in need thereof, the method comprising administering to the individual an expression vector comprising:
a. a first insert comprising a nucleic acid sequence encoding a Granulocyte Macrophage Colony Stimulating Factor (GM-CSF) sequence; and
b. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs,
wherein the virus uses a furin produced by the individual to enable infection of a cell of the individual by the virus, and
wherein the administering of the expression vector treats the respiratory condition.
   Embodiment 2. The method of embodiment 1, wherein administering the expression vector reduces or eliminates propagation of the virus causing the respiratory condition

Embodiment 3. The method of embodiment 1 or embodiment 2, wherein the second insert comprises a nucleic acid sequence according to SEQ ID NO: 2 or SEQ ID NO: 3.

Embodiment 4. The method of any one of embodiments 1-3, wherein the second insert comprises a nucleic acid sequence according to SEQ ID NO: 3.
Embodiment 5. The method of any one of embodiments 1-4, wherein the individual is a human.
Embodiment 6. The method of any one of embodiments 1-5, wherein the expression vector is a plasmid.
Embodiment 7. The method of any one of embodiments 1-6, wherein the expression vector is lyophilized with at least one stabilizing excipient prior to the administering, thereby producing lyophilized particles.
Embodiment 8. The method of embodiment 7, wherein the at least one stabilizing excipient is trehalose.
Embodiment 9. The method of embodiment 7 or embodiment 8, wherein the lyophilized particles are less than 5 μm in diameter.
Embodiment 10. The method of embodiment 9, wherein the lyophilized particles are from about 1 μm to 3 μm in diameter.
Embodiment 11. The method of any one of embodiments 1-10, wherein from about 1 mg to about 4 mg of the expression vector is administered to the individual.
Embodiment 12. The method of any one of embodiments 1-11, wherein the administering comprises pulmonary delivery.
Embodiment 13. The method of embodiment 12, wherein the administering comprises pulmonary delivery of the expression vector to the individual via a device selected from an inhaler or a nebulizer.
Embodiment 14. The method of any one of embodiments 1-13, wherein the virus comprises a glycoprotein requiring cleavage by the furin to allow entry of the virus into the cell of the individual.
Embodiment 15. The method of any one of embodiments 1-14, wherein the cell is an alveolar cell.
Embodiment 16. The method of any one of embodiments 1-15, wherein the virus causing the respiratory condition is a coronavirus.
Embodiment 17. The method of embodiment 16, wherein the coronavirus is a member selected from the group consisting of Severe Acute Respiratory Syndrome Coronavirus-2 (SARS-CoV-2), SARS-CoV and MERS-CoV.
Embodiment 18. The method of any one of embodiments 1-17, wherein the respiratory condition is Coronavirus disease 2019 (COVID-19).
Embodiment 19. The method of any one of embodiments 1-18, wherein the GM-CSF is a human GM-CSF sequence.
Embodiment 20. The method of any one of embodiments 1-19, wherein the expression vector further comprises a promoter.
Embodiment 21. The method of embodiment 20, wherein the promoter is a cytomegalovirus (CMV) mammalian promoter.
Embodiment 22. The method of embodiment 21, wherein the expression vector further comprises a CMV enhancer sequence and a CMV intron sequence.
Embodiment 23. The method of any one of embodiments 20-22, wherein the first insert and the second insert are operably linked to the promoter.
Embodiment 24. The method of any one of embodiments 1-23, wherein the expression vector further comprises a nucleic acid sequence encoding a picornaviral 2A ribosomal skip peptide between the first and the second nucleic acid inserts.
Embodiment 25. An inhalable dosage form, comprising:
a. an expression vector comprising
i. a first insert comprising a nucleic acid sequence encoding a Granulocyte Macrophage Colony Stimulating Factor (GM-CSF) sequence; and
ii. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
b. at least one stabilizing excipient.
   Embodiment 26. The inhalable dosage form of embodiment 25, wherein the second insert comprises a nucleic acid sequence according to SEQ ID NO: 2 or SEQ ID NO: 3.
   Embodiment 27. The inhalable dosage form of embodiment 25 or embodiment 26, wherein the second insert comprises a nucleic acid sequence according to SEQ ID NO: 3.
   Embodiment 28. The inhalable dosage form of any one of embodiments 25-27, wherein the at least one stabilizing excipient is trehalose.
   Embodiment 29. The inhalable dosage form of any one of embodiments 25-28, wherein the expression vector is a plasmid.
   Embodiment 30. The inhalable dosage form of any one of embodiments 25-29, wherein the inhalable dosage form comprises particles comprising the expression vector and the at least one stabilizing excipient.
   Embodiment 31. The inhalable dosage form of any one of embodiments 25-30, wherein the particles are less than 5 μm in diameter.
   Embodiment 32. The inhalable dosage form of embodiment 31, wherein the particles are from about 1 μm to 3 μm in diameter.
   Embodiment 33. The inhalable dosage form of any one of 30-32, wherein the particles are lyophilized particles.
   Embodiment 34. The inhalable dosage form of any one of embodiments 25-33, wherein the GM-CSF is a human GM-CSF sequence.
   Embodiment 35. The inhalable dosage form of any one of embodiments 25-34, wherein the expression vector further comprises a promoter.
   Embodiment 36. The inhalable dosage form of embodiment 35, wherein the promoter is a cytomegalovirus (CMV) mammalian promoter.
   Embodiment 37. The inhalable dosage form of embodiment 36, wherein the expression vector further comprises a CMV enhancer sequence and a CMV intron sequence.
   Embodiment 38. The inhalable dosage form of any one of embodiments 35-37, wherein the first insert and the second insert are operably linked to the promoter.
   Embodiment 39. The inhalable dosage form of any one of embodiments 25-38, wherein the expression vector further comprises a nucleic acid sequence encoding a picornaviral 2A ribosomal skip peptide between the first and the second nucleic acid inserts.
   Embodiment 40. The inhalable dosage form of any one of embodiments 25-39, wherein the lyophilized composition is formulated for pulmonary delivery.
   Embodiment 41. The inhalable dosage form of embodiment 40, wherein the lyophilized composition is formulated for pulmonary delivery via a device.
   Embodiment 42. The inhalable dosage form of embodiment 41, wherein the device is an inhaler or a nebulizer.
   Embodiment 43. A method of generating a lyophilized composition, the method comprising:

a. generating a liquid composition comprising:
i. an expression vector comprising
(a) a first insert comprising a nucleic acid sequence encoding a Granulocyte Macrophage Colony Stimulating Factor (GM-CSF) sequence; and
(b) a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
ii. at least one stabilizing excipient; and
b. lyophilizing the liquid composition from step a. using thin film freezing to generate lyophilized particles.
   Embodiment 44. The method of embodiment 43, wherein the second insert comprises a nucleic acid sequence according to SEQ ID NO: 2 or SEQ ID NO: 3.
   Embodiment 45. The method of embodiment 43 or embodiment 44, wherein the second insert comprises a nucleic acid sequence according to SEQ ID NO: 3.
   Embodiment 46. The method of any one of embodiments 43-45, wherein the at least one stabilizing excipient is trehalose.
   Embodiment 47. The method of any one of embodiments 43-46, wherein the liquid composition comprises from about 3% to 7% w/v of the at least one stabilizing excipient.
   Embodiment 48. The method of any one of embodiments 43-47, wherein the liquid composition comprises from about 5% w/v of the at least one stabilizing excipient.
   Embodiment 49. The method of any one of embodiments 43-48, wherein the expression vector is a plasmid.
   Embodiment 50. The method of any one of embodiments 43-49, wherein the lyophilized particles are less than 5 μm in diameter.
   Embodiment 51. The method of embodiment 50, wherein the lyophilized particles are from about 1 μm to 3 μm in diameter.
   Embodiment 52. The method of any one of embodiments 43-51, wherein the GM-CSF is a human GM-CSF sequence.
   Embodiment 53. The method of any one of embodiments 43-52, wherein the expression vector further comprises a promoter.
   Embodiment 54. The lyophilized composition of embodiment 53, wherein the promoter is a cytomegalovirus (CMV) mammalian promoter.
   Embodiment 55. The method of embodiment 54, wherein the expression vector further comprises a CMV enhancer sequence and a CMV intron sequence.
   Embodiment 56. The method of any one of embodiments 53-55, wherein the first insert and the second insert are operably linked to the promoter.
   Embodiment 57. The method of any one of embodiments 43-56, wherein the expression vector further comprises a nucleic acid sequence encoding a picornaviral 2A ribosomal skip peptide between the first and the second nucleic acid inserts.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a respiratory condition caused by a virus in an individual in need thereof, the method comprising administering to the individual an expression vector comprising:
a. a first insert comprising a nucleic acid sequence encoding a Granulocyte Macrophage Colony Stimulating Factor (GM-CSF) sequence; and
b. a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs,
wherein the virus uses a furin produced by the individual to enable infection of a cell of the individual by the virus, and
wherein the administering of the expression vector treats the respiratory condition.

2. The method of claim 1, wherein administering the expression vector reduces or eliminates propagation of the virus causing the respiratory condition

3. The method of claim 1, wherein the second insert comprises a nucleic acid sequence according to SEQ ID NO: 2 or SEQ ID NO: 3.

4. The method of claim 1, wherein the second insert comprises a nucleic acid sequence according to SEQ ID NO: 3.

5. The method of claim 1, wherein the individual is a human.

6. The method of claim 1, wherein the expression vector is a plasmid.

7. The method of claim 1, wherein the expression vector is lyophilized with at least one stabilizing excipient prior to the administering, thereby producing lyophilized particles.

8. The method of claim 7, wherein the at least one stabilizing excipient is trehalose.

9. The method of claim 7, wherein the lyophilized particles are less than 5 μm in diameter.

10. The method of claim 9, wherein the lyophilized particles are from about 1 μm to 3 μm in diameter.

11. The method of claim 1, wherein from about 1 mg to about 4 mg of the expression vector is administered to the individual.

12. The method of claim 1, wherein the administering comprises pulmonary delivery.

13. The method of claim 12, wherein the administering comprises pulmonary delivery of the expression vector to the individual via a device selected from an inhaler or a nebulizer.

14. The method of claim 1, wherein the virus comprises a glycoprotein requiring cleavage by the furin to allow entry of the virus into the cell of the individual.

15. The method of claim 1, wherein the cell is an alveolar cell.

16. The method of claim 1, wherein the virus causing the respiratory condition is a coronavirus.

17. The method of claim 16, wherein the coronavirus is a member selected from the group consisting of Severe Acute Respiratory Syndrome Coronavirus-2 (SARS-CoV-2), SARS-CoV and MERS-CoV.

18. The method of claim 1, wherein the respiratory condition is Coronavirus disease 2019 (COVID-19).

19. An inhalable dosage form, comprising:
a. an expression vector comprising
i. a first insert comprising a nucleic acid sequence encoding a Granulocyte Macrophage Colony Stimulating Factor (GM-CSF) sequence; and
ii. a second insert comprising a nucleic acid sequence encoding at a least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
b. at least one stabilizing excipient.

20. A method of generating a lyophilized composition, the method comprising:
a. generating a liquid composition comprising:
i. an expression vector comprising
(a) a first insert comprising a nucleic acid sequence encoding a Granulocyte Macrophage Colony Stimulating Factor (GM-CSF) sequence; and
(b) a second insert comprising a nucleic acid sequence encoding at least one bi-functional short hairpin RNA (bi-shRNA) capable of hybridizing to a region of a mRNA transcript encoding furin, thereby inhibiting furin expression via RNA interference wherein the bi-shRNA incorporates cleavage dependent siRNA and cleavage independent miRNA motifs; and
ii. at least one stabilizing excipient; and
b. lyophilizing the liquid composition from step a. using thin film freezing to generate lyophilized particles.
